SEQUENCE LISTING (6 sequences)

SEQ ID NO: 1 - DNA, 948 bp, Schizosaccharomyces pombe
atgaggaattcagtggaattctcacagttagggcttaaaacggcttttaatttatctcaaaataaaacttatacctcagcagtcaagaaacaattctttagtactggagcatttttaagcaatgggggaaatattgatttaaataaacacactcaaaagaacattgattcaaagtacgttgcttgcaactcaagatctgtaacacccccaaatgatgttgcttcttcagtttctaagaatacacttcgtcataaacaacgtttaatgatggctcaatggttaatgtctcccgaagtacagaaagcaaagtcctcatcatctggtaatgtaggcttgggacctaatacaaatcagcccttccctttaaaccctttttttaagcctcctaggcctatatctcattctttgcgtatgaaaattaccgatgaatacttgcagggtgcaagtatagaagtgcttgctcgaaaattcaacacatctccacaaaggattgaagctcttataaagttgagaagaataaatgatgaattcgaagaaaaaaagaagccgattcttcatagttataatgaagttatggagaaaatgctgaacgcatgtactaaacctgagatgatgcagtttaatgacggaaacgatattcctttgcgttccaatccagtatccctttggaagagtttgcctgagggagaaacgtttacacctcaagaggctgctaaaatcctgaagtggccttcgattgaggaattaaacatgagacaaaacgctactcacttccacaaaacatctgatgagcacaaagatttgaatgaagatgaagagctaatttcttcaagccccagcgaagttgggaagcgtgtttttaggctcattgatctttctactggtaatgtgtatagaagggataccggcggggatatttatgttaagagaaaaaaatctactacctga

SEQ ID NO: 2 - protein, 315 aa, Schizosaccharomyces pombe
MRNSVEFSQLGLKTAFNLSQNKTYTSAVKKQFFSTGAFLSNGGNIDLNKHTQKNIDSKYVACNSRSVTPPNDVASSVSKNTLRHKQRLMMAQWLMSPEVQKAKSSSSGNVGLGPNTNQPFPLNPFFKPPRPISHSLRMKITDEYLQGASIEVLARKFNTSPQRIEALIKLRRINDEFEEKKKPILHSYNEVMEKMLNACTKPEMMQFNDGNDIPLRSNPVSLWKSLPEGETFTPQEAAKILKWPSIEELNMRQNATHFHKTSDEHKDLNEDEELISSSPSEVGKRVFRLIDLSTGNVYRRDTGGDIYVKRKKSTT

SEQ ID NO: 3 - DNA, 1038 bp, Saccharomyces cerevisiae
  Intron (490)..(771)
atgagttacggcttaacaggtacctcttctaagctaagaggaactagttcaatattttcatggactcaggtgaggcacttttctcgtagaagaatagcctatccattttatccattcaagaaattgggaagacaacacccgaagaagcatgatacaaacttaaagactgctatgagacagtttttaggtccaaaaaattacaaaggagaatatgtcatgaacaagtattttacggttccgacgaatcatgtacctaactacattaagccagatttggaaaggggacaaagtttagaacacccggtaaccaagaagccattgcaactaaggtatgatgggacattaggtcctcctcctgtagaaaataaaaggttacaaaacattttcaaggatagattgttgcaacctttcccttcaaatccacattgtaagacgaattacgtattaagcccgcaattgaagcaaagcattttcgaagagattactgtggaaggactttctgcccaacaagtttctcaaaagtatggattaaaaattcctcgtgtagaggctattgttaaactggttagcgtggaaaacagctggaacagacggaatagagtatcttctgatttgaaaactatggacgagactttatatagaatgtttcccgtcttcgactcagatgcctcttttaaacgggagaatttaagcgaaattcctgttccgcaaaagactttggcgtcaagattcctcaccattgcagagtcagaaccttttgggcctgttgatgcagcccatgttttagaattagagcctgctgtagagaccttaaggaatttgtctactgtgggggaacattctagtggccatcaacaatccactaataagaatacgaaggttatatatggtgagcttgtcgaaggtgaaagatcacaatataaatttactaacgcaaaggttggaaaagtgggataccgttacggtagcggaaatagggataacaagaaagacagaagaattggctttaataaactgggccaaatggtatatatataa

SEQ ID NO: 4 - protein, 345 aa, Saccharomyces cerevisiae
MSYGLTGTSSKLRGTSSIFSWTQVRHFSRRRIAYPFYPFKKLGRQHPKKHDTNLKTAMRQFLGPKNYKGEYVMNKYFTVPTNHVPNYIKPDLERGQSLEHPVTKKPLQLRYDGTLGPPPVENKRLQNIFKDRLLQPFPSNPHCKTNYVLSPQLKQSIFEEITVEGLSAQQVSQKYGLKIPRVEAIVKLVSVENSWNRRNRVSSDLKTMDETLYRMFPVFDSDASFKRENLSEIPVPQKTLASRFLTIAESEPFGPVDAAHVLELEPAVETLRNLSTVGEHSSGHQQSTNKNTKVIYGELVEGERSQYKFTNAKVGKVGYRYGSGNRDNKKDRRIGFNKLGQMVYI

SEQ ID NO: 5 - DNA, 1227 bp, Candida albicans
atggttgtggggaagtctaatataatactgcagtcgagacatttgtcttctacttcaatatgcttaagattcaatagaagagcaacaatggacaagactgtgttgttgaacgaagctaaacagtttttcggaccaactaatgtcaaaggtgaacattgtaaaaacaaatttttctatccaccacaaaataatagaccaaattatatagtgaatgatggaagacctttagttggtgatcaattcccaacgaaaagaccaggaagaaattccaataatagagaaagaaaccctacagtacaccctttccctaataatatttacacgaaaactgcatacttgataccagaaaatataaaggataaaattgttgaagatgcaactactaatggattacatcctcaagaaattgctcataaatatagtataaatcttttaagagtagaagcgattttgaaattaagagatattgaaagcaaattcgttccagatgtatgtataaacatataaaaattgatttattaggatcacatatttaatgctgtgatttttgaattttgtttatttttttttggattgtgtttcaagaatgatgaaattgccatttttttatattcgattagtctttaagacaaacttacatggttataatttaagccctgaatttgaaacaccaacaaatgaaaaatctagcatttttaatatgttttttctgtttttttggtttttaataagaactcattttactaacaatgtttttttttggaattttaggaaaaaattgctgaagatttaaaccgatatgctaccattatgaagcgtatgttcccattatttaaaggtggatatagtgcagataatttgacggaaattccaactcctcataaaacattacaagatcgtttcttgaccattgaagaatcggaaccttttgggccagttgatgctgctagaatattgaaattagaaccagctgaagatacattgaagaaattgactgaatttgatgttgaacatgccaaggctcaacaagaagaattggatagaaagaaggttgatgttatttatggtaagagaagagaaggtgaaaaatcattatttaaattcaccatgaaagaagttggtaattttggttatcgttatggtgcctctagaagagatagaaagaaagatagagcaattggttttgacgcttctggtaagatggtctatctccaccctgaacaataa

SEQ ID NO: 6 - protein, 314 aa, Candida albicans
MVVGKSNIILQSRHLSSTSICLRFNRRATMDKTVLLNEAKQFFGPTNVKGEHCKNKFFYPPQNNRPNYIVNDGRPLVGDQFPTKRPGRNSNNRERNPTVHPFPNNIYTKTAYLIPENIKDKIVEDATTNGLHPQEIAHKYSINLLRVEAILKLRDIESKFVPDEKIAEDLNRYATIMKRMFPLFKGGYSADNLTEIPTPHKTLQDRFLTIEESEPFGPVDAARILKLEPAEDTLKKLTEFDVEHAKAQQEELDRKKVDVIYGKRREGEKSLFKFTMKEVGNFGYRYGASRRDREKDRAIGFDASGKMVYLHPEQ

(19) United States
(12) Patent Application Publication
(10) Pub. No.: US 2004/0106173 A1
(43) Pub. Date: Jun. 3, 2004

(54) Bot1: target for antifungal agents

(75) Inventors: Fulvia Verde, Miami Beach, FL (US); Paola Catanuto, North Miami, FL (US); David J. Wiley, Miami, FL (US); Min You, Miami, FL (US)

Correspondence Address: NIXON & VANDERHYE P.C., 8th Floor, 1100 North Glebe Road, Arlington, VA, 22201-4714, US

(73) Assignee: The University of Miami

(21) Appl. No.: 10/174,487

(22) Filed: Jun. 19, 2002

Related U.S. Application Data
(60) Provisional application No. 60/298,901, filed on Jun. 19, 2001.

(51) Int. Cl. C12Q 1/68; C07H 21/04; C12N 9/24; C12N 1/18

(52) U.S. Cl. 435/69.1; 435/200; 435/320.1; 435/254.2; 536/23.2; 435/6

(57) ABSTRACT

We have discovered a target protein which is conserved in fungi and essential for cell viability, cell growth, the control of cell morphogenesis, and combinations thereof. An antifungal agent targets the protein in a fungus, but not an infected host. This protein is encoded by the bot1 gene in Schizosaccharomyces pombe, Saccharomyces cerevisiae, and Candida albicans but is not detected in a filamentous fungus.

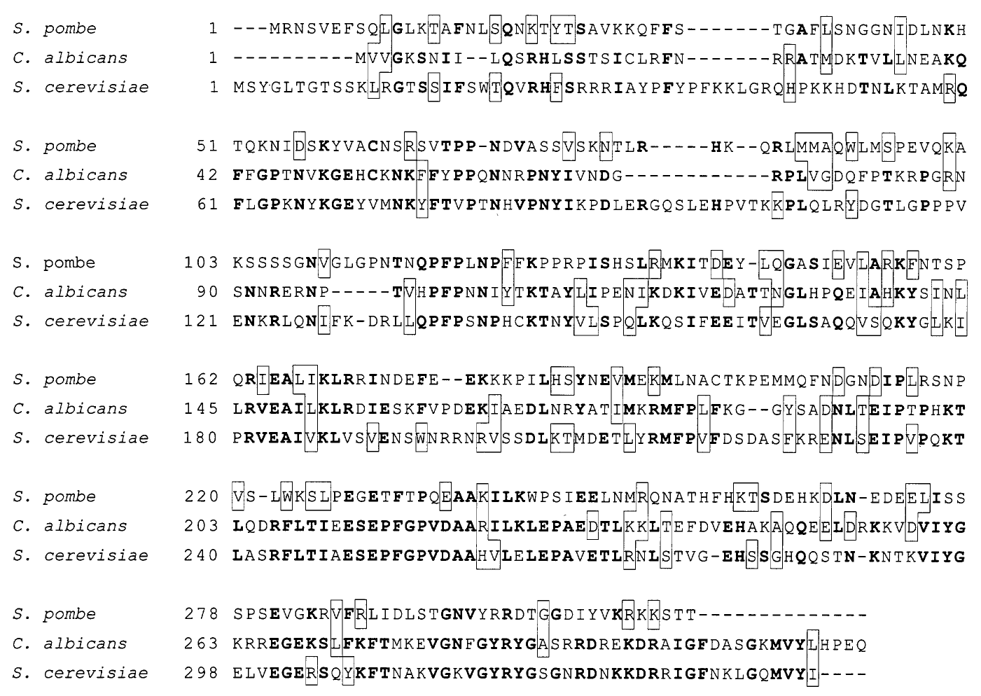

### CROSS-REFERENCE TO RELATED APPLICATIONS

This application claims benefit of provisional Appln. No. 60/298,901, filed Jun. 19, 2001.

### BACKGROUND OF THE INVENTION

This invention relates to a target protein conserved in fungi and required for cell viability, cell growth, the control of cell morphogenesis, or combinations thereof. This protein is encoded by the bot1 gene in Schizosaccharomyces pombe, Saccharomyces cerevisiae, and Candida albicans.

Fungal pathogens are responsible for diseases of humans, animals, and plants. Fungal diseases often occur as opportunistic infections in humans with suppressed immune systems, such as patients afflicted by AIDS, leukemia, or diabetes mellitus or patients receiving immunosuppressive drugs or chemotherapy. Fungal infections are a significant problem in veterinary medicine as well, and fungal diseases also affect plant crops which are critical to the agricultural industry. Since fungi are eukaryotic cells, many metabolic pathways and genes of fungi are similar to those of mammalian and/or plant cells. Therefore, treatment of fungal diseases is frequently hindered because antifungal agents are toxic to the host.

A fungal infection may be cutaneous, subcutaneous, or systemic. Superficial mycoses include tinea capitis, tinea corporis, tinea pedis, perionychomycosis, pityriasis versicolor, oral thrush, and other candidoses such as vaginal, respiratory tract, biliary, easophageal, and urinary tract candidoses. Systemic mycoses include systemic and mucocutaneaus candidosis, cryptococcosis, aspergillosis, mucormycosis (phycomycosis), paracoccidioidomycosis, North American blastomycosis, histoplasmosis, coccidioidomycosis, and sporotrichosis. Fungal infections can also contribute to meningitis and pulmonary or respiratory tract diseases. Opportunistic fungal infections proliferate, especially in patients afflicted with AIDS or other diseases that compromise the immune system.

Fungal infection is also a significant problem in veterinary medicine. Some of the fungi that infect animals can be transmitted from animals to humans. Fungal infections or infestations are also a very serious problem in agriculture with fungicides being employed to protect vegetable and fruit and cereal crops. Fungal attack of wood products is also of major economic importance. Additional products that are susceptible to fungal infestation include textiles, plastics, paper and paint.

The use of herbicides and insecticides are critical in agriculture to ensure an adequate food supply for a growing world population. One problem with current herbicides and insecticides is that agricultural pests often become resistant to them. Another problem is that many pesticides currently in use are highly toxic to farm workers in the fields, humans or animals who eat the food produced by the treated crops, and other plant and animal species that come in contact with the pesticide through soil, water or air contamination. Thus, new herbicides and insecticides that are less toxic to humans and animals and that are effective against resistant species of weeds and insects are desirable.

Examples of pathogenic fungi include dermatophytes (e.g., Microsporum canis and other M. spp.; and Trichophyton spp. such as T. rubrum, and T. mentagrophytes), yeasts (e.g., Candida albicans, C. Tropicalis, or other Candida species), Torulopsis glabrata, Epidermophyton floccosum, Malassezia furfur (Pityropsporon orbiculare, or P. ovale), Cryptococcus neoformans, Aspergillus fumigatus, and other Aspergillus spp., Zygomycetes (e.g., Rhizopus, Mucor), Paracoccidioides brasiliensis, Blastomyces dermatitides, Histoplasma capsulatum, Coccidioides immitis, and Sporothrix schenckii.

By way of background, the Fungi Kingdom consists of two divisions, the Eumycota and Myxomycota or the true fungi and slime molds, respectively. The true fungi are those species that are hyphal or are clearly related to species that are hyphal, possess cell walls throughout most or all of their life cycle, and are exclusively absorptive in their function. The slime molds are organisms that do not form hyphae, lack cell walls during the phase in which they obtain nutrients and grow and are capable of ingesting nutrients in particulate form by phagocytosis.

The two most important classes of true fungi in which most species produce motile cells, known as zoospores, are the Oomycetes, and the Chytridiomycetes. The fungi that lack zoospores are classified according to the sexual phase of the fungal life cycle. The sexual process leads to the production of characteristic spores in the different groups. The fungi that form zygospores are classified as Zygomycetes, those that form ascospores are classified as Ascomycetes, and those fanning basidiospores are classified as Basidiomycetes. There are also many species, recognizable as higher fungi through the presence of cell walls in their hyphae, that produce asexual spores but lack a sexual phase. These are known as Deuteromycetes, and details of their asexual sporulation are used to classify them. A representative member of the Deuteromycetes includes Candida albicans.

Yeast are fungi that are normally unicellular and reproduce by budding although some will, under appropriate conditions, produce hyphae, just as some normally hyphal fungi may produce a yeast phase. Diploid yeast may mate following ascospore germination, but single cells can be used to establish permanently haploid cultures.

By way of example, the yeast Candida albicans has been shown to be one of the most pervasive fungal pathogens in humans. It has the capacity to opportunistically infect a diverse spectrum of compromised hosts, and to invade many diverse tissues in the human body. In many instances, it can evade antibiotic treatment and the immune system. Although C. albicans is a member of the normal flora of the mucous membranes in the respiratory, gastrointestinal, and female genital tracts, it may gain dominance and be associated with pathologic conditions in such locations. Sometimes it produces progressive systematic disease in debilitated or immunosuppressed patients (e.g., sepsis), particularly if cell-mediated immunity is impaired. Candida may produce bloodstream invasion, thrombophlebitis, endocarditis, or infection of the eyes and virtually any organ or tissue when introduced intravenously: for example, via tubing, needles, narcotics abuse, etc.

Candida albicans has been shown to be diploid and, therefore, probably does not go through a sexual phase or meiotic cycle. This yeast appears to be able to spontaneously and reversibly switch at high frequency between at least seven phenotypes. Switching has been shown to occur not only in standard laboratory strains, but also in strains isolated from the mouths of healthy individuals.

Nystatin, ketoconazole, and amphotericin B are drugs that have been used to treat oral and systemic Candida infections. But orally administered nyastin is limited to treatment within the gut and is not applicable to systemic treatment. Some systemic infections are susceptible to treatment with ketoconazole or amphotericin B, but these drugs may not be effective in such treatment unless combined with additional drugs. Amphotericin B has a relatively narrow therapeutic index and numerous undesirable side effects, ranging from nausea and vomiting to kidney damage and toxicities occur even at therapeutic concentrations. While ketoconazole and other azole antifungals exhibit significantly lower toxicity, their mechanism of action, through inactivation of cytochrome P₄₅₀ prosthetic group in certain enzymes (some of which are found in humans) precludes use in patients that are simultaneously receiving other drugs that are metabolized by the body's cytochrome P₄₅₀ enzymes. These adverse effects mean that their use is generally limited to the treatment of topical or superficial infections. In addition, resistance to these compounds is emerging and may pose a serious problem in the future. The more recently developed triazole drugs, such as fluconazole, are believed by some to have fewer side effects but are not completely effective against all pathogens. Fungicide resistance generally develops when a fungal cell or fungal population that originally was sensitive to a fungicide becomes less sensitive by heritable changes after a period of exposure to the fungicide.

Because no single approach may be effective against all fungal pathogens and because of the possibility of developed resistance to previously effective antifungal compounds, there remains a need for new antifungal agents with novel mechanisms of action and improved or different activity profiles. There is also a need for agents which are active against fungi but are not toxic to mammalian cells, as toxicity to mammalian cells can lead to a low therapeutic index and undesirable side effects in the host (e.g., a patient). An important aspect of meeting this need is the selection of an appropriate component of fungal structure or metabolism as a therapeutic target.

Some drug discovery efforts have been directed at components of the fungal cell or metabolic pathways which are unique to fungi, and hence might be used as targets of new therapeutic agents. Preferably, these should act on the fungal pathogen without undue toxicity to host cells. Because no single approach is effective against all fungal pathogens and because of the possibility of developed resistance to previously effective antifungal agents, there remains a need for new antifungal agents with novel mechanisms of action. An essential aspect of meeting this need is the selection of an appropriate component of fungal structure or metabolism as a therapeutic target.

Despite the increased use of rational drug design, a preferred method continues to be the mass screening of compound libraries for active agents by exposing cultures of pathogens to the test compounds and assaying for inhibition of growth. In testing thousands or tens of thousands of compounds, however, a correspondingly large number of fungal cultures must be grown over time periods which are relatively long. Moreover, a compound which is found to inhibit fungal growth in culture may be acting not on the desired target but on a different, less unique fungal component, with the result that the compound may act against host cells as well and thereby produce unacceptable side effects. Consequently, there is a need for screening methods which more specifically identify those agents that are active against a certain cellular target.

It is an objective of the invention to provide a novel target gene and its encoded protein for antifungal agents. The need for a target that is expressed in fungi, but not in their infected host, is thereby addressed. Other advantages and improvements are discussed below, or would be apparent from the disclosure herein.

Polynucleotides, polypeptides, transfected and mutant fungi, and methods for using and making the aforementioned products are provided herein. Further objectives and advantages of the invention are described below.

### SUMMARY OF THE INVENTION

An object of the invention is to provide polynucleotides corresponding to a bot1 gene and polypeptides corresponding to the Bot1 protein encoded thereby. This includes the nucleotide and amino acid sequences listed herein, those containing mutations, and other variants thereof (e.g., partial-length oligonucleotides and oligopeptides). Hybrids between at least one Bot1 portion and a heterologous portion (polynucleotide or polypeptide) are chimeric gene or fusion protein variants, respectively. Constructs may be used to shuttle at least one Bot1 portion into a host or to express at least one Bot1 portion by transcription and/or translation in a host or using at least partially purified components (e.g., a cell-free or membrane extract). Cells (e.g., fungi with a mutated bot1 genetic locus or ectopic bot1 gene), extracts thereof containing the bot1 gene and/or Bot1 protein, and complexes formed with Bot1 protein are also provided.

Another object of the invention is to provide processes for transcribing at least a bot1 gene or fragment thereof, translating at least a Bot1 transcript or fragment thereof, producing at least a Bot1 protein or fragment thereof with an expression construct in a host, recombining at least one Bot1 portion and a heterologous portion, shuttling at least one Bot1 portion into a host, directly or indirectly immobilizing Bot1 or a fragment thereof to a substrate, extracting a cell, and the products made thereby. These products may then be subjected to further processing (e.g., Bot1-specific binding by nucleic acid hybridization or protein-specific complex formation, detecting a quantifiable amount or at least the presence of Bot1, identifying a previously known or unknown variety of Bot1, isolating Bot1 from a mixture or library, direct or indirect labeling of Bot1, purifying Bot1 from contaminants, or combinations thereof).

Yet another object of the invention is to provide a process for assaying one or more activities of a polynucleotide corresponding to a fungal bot1 gene, a polypeptide corresponding to Bot1 protein encoded thereby, or fragments thereof. For example, the activity may be cell viability, cell growth, the control of cell morphogenesis, or combinations thereof. An antifungal agent may be screened, selected, or validated as effective, improved, or specific.

Also provided are processes for using and making the aforementioned products. Further aspects of the invention will be apparent to a person skilled in the art from the following detailed description and claims, and generalizations thereto.

### BRIEF DESCRIPTION OF THE DRAWING

The FIGURE shows an alignment of Bot1p and homologous proteins. A BLAST homology search of EMBL and GenBank databases (NCBI) using default parameters identified Saccharomyces cerevisiae YGR165w and Candida albicans (gnl|SDSTC 5476|C. albicans Contig5-3220) as homologous to Schizosaccharomyces. pombe Bot1p (AF352796). Sequences were aligned to minimize the introduction of gaps and to maximize the number of identical or similar amino acid residues. Identical amino acid residues are in bold and similar amino acid residues (i.e., a conservative change) are boxed. Sequences of the S. pombe gene and protein (SEQ ID NOS:1-2, respectively), the S. cerevisiae gene and protein (SEQ ID NOS:3-4, respectively), and the C. albicans gene and protein (SEQ ID NOS:5-6, respectively) are provided in the Sequence Listing.

### DESCRIPTION OF SPECIFIC EMBODIMENTS OF THE INVENTION

In eukaryotic cells, the microtubule cytoskeleton has a fundamental role in the establishment of spatial organization. We were interested in increasing understanding of this process at the molecular level. In S. pombe, a pivotal component of the spatial control of cell morphogenesis is Tea1p, which is delivered to the cell surface in a microtubule-dependent fashion. We have identified a novel protein Bot1p which associates with Tea1p and plays a role in cell morphogenesis. Intriguingly, Bot1p is required for the normal localization of Orb6p, a protein kinase essential for the maintenance of cell polarity and related to human Ndr kinase, C. elegans Sax1 and S. cerevisiae Cbk1p. Bot1p associates with Orb6p by two-hybrid analysis and co-purification studies. Our results indicate that Tea1p controls Bot1p localization to the cell tips. Bot1p then participates in the establishment of polarized cell growth by associating with Orb6p. These findings indicate how Tea1p may function as a positional marker for cell polarity and provide insights into the mechanism of spatial localization of a conserved kinase Orb6p.

Polynucleotides representative of bot1 genes, which include mutants and other variants thereof, may be used to identify, isolate, or detect complementary polynucleotides by binding assays. Similarly, polypeptides representative of Bot1 proteins, which include mutants and other variants thereof, may be used to identify, isolate, or detect interacting proteins by binding assays. Optionally, bound complexes including interacting proteins (with or without Tea1 protein and/or Orb6 protein) may be identified, isolated, or detected indirectly though a binding molecule (e.g., antibody, natural or normatural peptide mimetic) for the Bot1 gene product. Interacting proteins may also be targets of antifungal agents. Affinity chromatography of DNA-binding proteins, electrophoretic mobility shift assay (EMSA), one- or two-hybrid system, membrane protein cross-linking, and screening a phage display library may be used for identifying, isolating, or detecting interacting proteins. Candidate compounds useful for treating fungal disease may interact with a representative polynucleotide or polypeptide, and be screened for their ability to provide therapy or prophylaxis. These products may be used in assays (e.g., diagnosis) or for treatment; conveniently, they are packaged as assay kits or in pharmaceutical form.

Another aspect of the invention is a hybrid Bot1 polynucleotide or polypeptide: e.g., a transcriptional chimera or a translational fusion. In transcriptional chimeras, at least a transcriptional regulatory region of a heterologous gene is ligated to a Bot1 polynucleotide or, alternatively, a transcriptional regulatory region of a bot1 gene is ligated to at least a heterologous polynucleotide. The reading frames of a Bot1 polypeptide and at least a heterologous amino acid domain are joined in register for a translational fusion. If a reporter or selectable marker is used as the heterologous region or domain, then the effect of mutating Bot1 nucleotide or amino acid sequences on Bot1 function may be readily assayed. In particular, a transcriptional chimera may be used to localize a regulated promoter of a bot1 gene and a translational fusion may be used to localize Bot1 protein in the cell. Sequence specificity may be changed or conferred by joining a Bot1 polypeptide to a heterologous DNA-binding domain (DBD) of known sequence specificity.

“Bot1” refers to binding component of Tea1p, other native (i.e., derived from nature) genes and proteins, and variant forms thereof (e.g., mutants and analogs not found in nature). The chemical structure of Bot1 may be a polymer of natural or non-natural nucleotides connected by natural or normatural covalent linkages (i.e., polynucleotide) or a polymer of natural or normatural amino acids connected by natural or normatural covalent linkages (i.e., polypeptide). See Tables 1-4 of WIPO Standard ST.25 (1998) and M.P.E.P. § 2422 for a nonlimiting list of natural and normatural nucleotides and amino acids.

The natural linkage for polynucleotides is a phosphodiester bond made between the 3′ hydroxyl group of one nucleotide and the 5′ phosphate group of the succeeding nucleotide. Normatural backbones that include a phosphorus heteroatom are phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Normatural backbones that do not include a phosphorus heteroatom are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide, and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ groups. Post-transcriptional modifications include polyadenylation and splicing.

Various modifications to the polynucleotides can be introduced to increase stability and half-life. Possible modifications include, but are not limited to, the addition of flanking sequences of ribonucleotides or deoxynucleotides to the 5′ and/or 3′ ends, blocking or cyclization of the 5′ and/or 3′ ends, or the use of phosphorothioate or 2′-O-methyl rather than phosphodiesterase linkages within the backbone.

The natural linkage for polypeptides is an amide bond made between the α-carboxyl group of the N-terminal residue and the α-amino group of the C-terminal residue. Backbones may be modified by using D-amino acids, modified amino acids, and peptidomimetics. Nonpeptide linkages between amino acid residues may include —CH₂NH—, —CH₂S—, —CH₂—CH₂-, —CH═CH— (cis or trans), —COCH₂—, —CH(OH)CH₂—, —CH₂SO—, and others having mixed P, O, Si, S and CH₂ groups.

“Mutants” are polynucleotides and polypeptides having at least one function that is more active or less active, an existing function that is changed or absent, a novel function that is not naturally present, or combinations thereof. “Analogs” are polynucleotides and polypeptides with different chemical structure, but substantially equivalent function as compared to the native gene or protein. Bot1 functions are described in detail herein. Mutants and analogs can be made by genetic engineering or chemical synthesis, but the latter is preferred for normatural nucleotides, amino acids, or linkages.

“Oligonucleotides” and “oligopeptides” are short versions of polynucleotides and polypeptides (e.g., less than 50 or 100 nucleotides or amino acids). Generally, they can be made by chemical synthesis, but cleavage of longer polynucleotides or polypeptides can also be used. Electrophoresis and/or reverse phase high-performance liquid chromatography (HPLC) are suitable biochemical techniques to purify short products.

“Fungal Bot1” means a Bot1 derived from a fungus and includes polymorphic and mutant versions thereof, but excludes similar genes and proteins derived from other organisms. A bot1 gene can be isolated using stringent hybridization: e.g., 400 mM NaCl, 40 mM PIPES pH 6.4, 1 mM EDTA, 50° C. or 70° C. for oligonucleotides; 500 mM NaHPO₄ pH 7.2, 7% sodium dodecyl sulfate (SDS), 1% bovine serum albumin (BSA, fraction V), 1 mM EDTA, 45° C. or 65° C. for polynucleotides of 50 bases or longer. Bot1 proteins can be isolated using antibody or other binding protein (e.g., immunoblotting): suitable conditions could be 50 mM Tris-HCl pH 7.4, 500 mM NaCl, 0.05% TWEEN 20 surfactant, 1% bovine serum albumin (BSA, fraction V), room temperature. Washing conditions may be varied by adjusting salt concentration and temperature such that the signal-to-noise ratio is sufficient for specific binding. Such isolation methods may be used to identify an unknown fungal Bot1-related nucleic acid or protein using a probe which specifically binds a known fungal Bot1 nucleic acid or protein, respectively. For example, a mixture of nucleic acids or proteins may be separated by one or more physical, chemical, and/or biological properties, and then the presence or absence of Bot1 nucleic acid or protein may be detected by specific binding of the probe. The probe may also be used to detect the presence or absence of a known fungal bot1 gene or Bot1 protein. Blocking and washing conditions can be varied to obtain a nucleic acid hybridization or protein binding signal that is target specific and/or reduces the background.

An “isolated” product is at least partially purified from its cell of origin (e.g., human, other mammal, bacterium, yeast). For example, as compared to a lysate of the cell of origin, the isolated product is at least 50%, 75%, 90%, 95% or 98% purified from other chemically-similar solutes (e.g., nucleic acids for polynucleotides, nucleoproteins for polypeptides). For a chemically-synthesized polymer of nucleotides or amino acids, purity is determined by comparison to prematurely terminated or blocked products and may, as a practical matter, be considered isolated without purification. Purification may be accomplished by biochemical techniques such as, for example, antibody or solvent precipitation, cell fractionation, centrifugation, chromatography, electrophoresis, or combinations thereof. Generally, solvent (e.g., water) and functionally inert chemicals like buffers and salts are disregarded when calculating purity. Cloning is often used to isolate the desired product.

The meaning of “heterologous” depends on context. For example, ligation of heterologous nucleotide regions to form a chimera means that the regions are not found colinear in nature (e.g., a fungus-derived Bot1 polynucleotide ligated to a fungal non-Bot1 transcriptional regulatory region). Another example is that fusion of heterologous amino acid domains means that the domains are not found colinear in humans (e.g., a Bot1 polypeptide joined to a non-Bot1 dimerization domain). Ligation of nucleotide regions or joining of amino acid domains, one derived from a fungus and another derived from a nonfungus, are heterologous because they are derived from different species. In a further example, transfection of a vector or expression construct into a heterologous host cell or transgenesis of a heterologous nonhuman organism means that the vector or expression construct is not found in the cell's or organism's genome in nature. A “recombinant” product is the result of ligating heterologous regions for a recombinant polynucleotide or fusing heterologous domains for a recombinant polynucleotide. Recombination may be genetically engineered in vitro with purified enzymes or in vivo in a cultured cell. The bot1 gene maps to chromosome 2 of S. pombe. A gene or expressed portion thereof (e.g., exon or transcribed region) adjoining bot1 may be excluded from a recombinant polynucleotide.

According to one aspect of invention, polynucleotides (e.g., DNA or RNA, single- or double-stranded) that specifically hybridize to bot1 genes and transcripts thereof can be used as probes or primers. Such polynucleotides could be full length covering the entire gene or transcribed message (e.g., a recombinant clone in a phagemid, plasmid, bacteriophage, cosmid, shuttle vector, yeast artificial chromosome or YAC, bacterial artificial chromosome or BAC, or other vector), a particular coding region, or a shorter length sequence which is unique to bot1 genes or transcripts thereof but contains only a portion of same. A probe stably binds its target to produce a hybridization signal specific for a Bot1 polynucleotide or polypeptide, while a primer may bind its target less stably because repetitive cycles of polymerization or ligation will also produce a specific amplification signal. The polynucleotide may be at least 15, 30, 45, 60, 90, 120, 240, 360, 480, 600, 720, 1200, 2400, 5000, 10K, 20K, 40K, 100K, 250K, or 500K nucleotides long (including intermediate ranges thereof).

Typically, a nucleotide sequence may show as little as 85% sequence identity, and more preferably at least 90% sequence identity compared to SEQ ID NO:1, 3 or 5, excluding any deletions or additions which may be present, and still be considered related. Nucleotide sequence identity may be at least 95% and, more preferably, nucleotide sequence identity is at least 98%. Amino acid sequences are considered to be related with as little as 90% sequence identity compared to SEQ ID NO:2, 4 or 6. But 95% or greater sequence identity is preferred and 98% or greater sequence identity is more preferred.

Use of complex mathematical algorithms is not required if amino acid sequences can be aligned without introducing many gaps. But such algorithms are known in the art, and implemented using default parameters in commercial software packages provided by DNASTAR, Genetics Computer Group, Hitachi Genetics Systems, and Oxford Molecular Group. See Doolittle, Of URFS and ORFS, University Science Books, 1986; Gribskov and Devereux, Sequence Analysis Primer, Stockton Press, 1991; and references cited therein. Percentage identity between a pair of sequences may be calculated by the algorithm implemented in the BESTFIT computer program (Smith and Waterman, J. Mol. Biol., 147:195-197, 1981; Pearson, Genomics, 11:635-650, 1991). Another algorithm that calculates sequence divergence has been adapted for rapid database searching and implemented in the BLAST computer program (Altschul et al., Nucl. Acids Res., 25:3389-3402, 1997).

Conservative amino acid substitutions, such as Glu/Asp, Val/Ile, Ser/Thr, Arg/Lys and Gln/Asn (see also the substitutions boxed in the FIGURE), may also be considered when making comparisons because the chemical similarity of these pairs of amino acid residues would be expected to result in functional equivalency. Amino acid substitutions that are expected to conserve the biological function of the polypeptide would conserve chemical attributes of the substituted amino acid residues such as hydrophobicity, hydrophilicity, side-chain charge, or size. Functional equivalency or conservation of biological function may be evaluated by methods for structural determination and bioassay as disclosed herein. Thus, amino acid sequences are considered to be related with as little as 90% sequence similarity between the two polypeptides; however, 95% or greater sequence similarity is preferred and 98% or greater sequence similarity is most preferred.

The codons used in the native nucleotide sequences may be adapted for translation in a heterologous host by adopting the codon preferences of the host. This would accommodate the translational machinery of the heterologous host without a substantial change in the chemical structure of the polypeptide.

Bot1 polypeptide and its variants (i.e., deletion, domain shuffling or duplication, insertion, substitution, or combinations thereof) are useful for determining structure-function relationships (e.g., alanine scanning, conservative or nonconservative amino acid substitution). See Wells (Bio/Technology, 13:647-651, 1995) and U.S. Pat. No. 5,534,617. Directed evolution by random mutagenesis or gene shuffling using Bot1. Mutant and analog Bot1 polypeptides are encoded by suitable mutant and analog Bot1 polynucleotides.

Structure-activity relationships of Bot1 may be studied (i.e., SAR studies) using variant polypeptides produced by an expression construct transfected in a host cell with or without endogenous Bot1. Thus, mutations in discrete domains of the Bot1 polypeptide may be associated with decreasing or even increasing activity in the protein's function.

A Bot1 nucleotide sequence can be used to produce a fusion polypeptide with at least one heterologous peptide domain (e.g., an affinity or epitope tag). Oligopeptide is useful for producing specific antibody and epitope mapping of Bot1-specific antibody. A polypeptide may be at least 10, 15, 20, 25, 30, 35, 40, 45, 50, 60, 70, 80, 90, 100, 150, or more amino acids long (including intermediate ranges thereof). Oligopeptide may be conjugated to one affinity tag of a specific binding pair (e.g., antibody-digoxygenin/hapten/peptide, biotin-avidin/streptavidin, glutathione S transferase-glutathione, maltose binding protein-maltose, polyhistidine-nickel, protein A or G/immunoglobulin). Either a full-length Bot1 polypeptide (SEQ ID NO:2, 4 or 6) or a shorter fragment unique to the Bot1 amino acid sequence can be produced; optionally including a heterologous peptide domain. The latter oligopeptides have been used to raise antiserum. Bot1 polypeptide may be synthesized by chemical means, purified from natural sources, synthesized in transfected host cells, or combinations thereof.

The Bot1 nucleotide sequence or a portion thereof can be used as a probe to monitor bot1 gene rearrangement and/or expression. The invention also provides hybridization probes and amplification primers (e.g., polymerase chain reaction or PCR, ligation chain reaction or LCR, other isothermal amplification reactions). A pair of such primers has been used for RT-PCR assays to quantitate Bot1 transcript abundance within cells. Amplification primers may be between 15 and 30 nucleotides long (preferably about 25 nucleotides), anneal to either sense or antisense strand (preferably the pair will be complementary to each strand), and terminate at the 3′ end anywhere within SEQ ID NOS:1, 3 and 5 or their complements. Therefore, this invention will be useful for development and utilization of Bot1 primers and other oligonucleotides to quantitate cognate RNA and DNA within cells.

Assaying Polynucleotides or Polypeptides

Binding of polynucleotides or polypeptides may take place in solution or on a substrate. The assay format may or may not require separation of bound from not bound. Detectable signals may be direct or indirect, attached to any part of a bound complex, measured competitively, amplified, or any combination thereof. A blocking or washing step may be interposed to improve sensitivity and/or specificity. Attachment of a polynucleotide or polypeptide, interacting protein, or binding molecule to a substrate before, after, or during binding results in capture of an unattached species. Such immobilization will be stably attached to the substrate under washing conditions. See U.S. Pat. Nos. 5,143,854 and 5,412,087.

Polynucleotide, polypeptide, or binding molecule may be attached to a substrate. The substrate may be solid or porous and it may be formed as a sheet, bead, fiber, tape, tube, or wire. The substrate may be made of cotton, silk, or wool; cellulose, nitrocellulose, nylon, or positively-charged nylon; natural, butyl, silicone, or styrenebutadiene rubber; agarose or polyacrylamide; crystalline silicon or polymerized organosiloxane; crystalline, amorphous, or impure silica (e.g., quartz) or silicate (e.g., glass); polyacrylonitrile, polycarbonate, polyethylene, polymethyl methacrylate, polymethylpentene, polypropylene, polystyrene, polysulfone, polytetrafluoroethylene, polyvinylidenefluoride, polyvinyl acetate, polyvinyl chloride, or polyvinyl pyrrolidone; or combinations thereof. Optically-transparent materials are preferred so that binding can be monitored and any signal can be transmitted by light. For example, one or more beads suspended in solution and at the end of an optical fiber can be interrogated by a light signal (e.g., blue, red, or green) sent through the optical fiber when an analyte in solution (e.g., probe conjugated to a blue, red, or green label) binds to the bead, which is attached to the polynucleotide, polypeptide, or binding molecule. Such reagents would allow capture of a molecule in solution by specific binding, and then interaction of the molecule with and immobilization to the substrate. Monitoring gene expression is facilitated by using an ordered substrate array or coded library of multiple substrates.

Polynucleotide, polypeptide, or binding molecule may be synthesized in situ by solid-phase chemistry or photolithography to directly attach the nucleotides or amino acids to the substrate. Attachment of the polynucleotide, polypeptide, or binding molecule to the substrate may be through a reactive group as, for example, a carboxy, amino, or hydroxy radical; attachment may also be accomplished after contact printing, spotting with a pin, pipetting with a pen, or spraying with a nozzle directly onto a substrate. Alternatively, the polynucleotide, polypeptide, or binding molecule may be reversibly attached to the substrate by interaction of a specific binding pair (e.g., antibody-digoxygenin/hapten/peptide epitope, biotin-avidin/streptavidin, glutathione S transferase or GST-glutathione, lectin-sugar, maltose binding protein-maltose, polyhistidine-nickel, protein A/G-immunoglobulin); cross-linking may be used if irreversible attachment is desired.

By synthesizing the polynucleotide, polypeptide, or binding molecule in situ or otherwise attaching it to a substrate at a predetermined, discrete position or to a coded substrate, an interacting polynucleotide, polypeptide, or specific binding molecule can be identified without determining its sequence. For example, a polynucleotide, polypeptide, or binding molecule of known sequence can be determined by its position (e.g., rectilinear or polar coordinates) or decoding its signal (e.g., combinatorial tag, electromagnetic radiation) on the substrate. A nucleotide or amino acid sequence will be correlated with each position on or decoded signal of the substrate. A substrate may have a pattern of different polynucleotides, polypeptides, and/or binding molecules (e.g., at least 5, 10, 20, 30, 40, 50, 60, 80, 100, 150, 200, 250, 300, 350, 400, 450, 500, 1000, 2000, 3000, 4000, 5000, 7500, 10,000, 50,000, 100,000 or 1,000,000 distinguishable positions) at low or high density (e.g., at least 1,000, 10,000, 100,000 or 1,000,000 distinguishable positions per cm²). The number of sequences that can be differentiated by the signal is only limited by factors such as the number and complexity of combinations; interference between a property of electromagnetic radiation like wavelength, frequency, energy, polarization; etc.

Multiplex analysis may be used to monitor expression of different genes at the same time in parallel. Such multiplex analysis may be performed using different polynucleotides, polypeptides, or binding molecules arranged in high density on a substrate. Simultaneous solution methods such as multiprobe ribonuclease protection assay or multiprimer pair amplification associate each transcript with a different length of detected product which is resolved by separation on the basis of molecular weight.

Changes in gene expression may be manifested in the cell by affecting transcriptional initiation, transcript stability, translation of transcript into protein product, protein stability, or combinations thereof. The gene, transcript, or polypeptide can also be assayed by techniques such as in vitro transcription, in vitro translation, Northern hybridization, nucleic acid hybridization, reverse transcription-polymerase chain reaction (RT-PCR), run-on transcription, Southern hybridization, cell surface protein labeling, metabolic protein labeling, antibody binding, immunoprecipitation (IP), enzyme linked immunosorbent assay (ELISA), electrophoretic mobility shift assay (EMSA), radioimmunoassay (RIA), fluorescent or histochemical staining, microscopy and digital image analysis, and fluorescence activated cell analysis or sorting (FACS).

A reporter or selectable marker gene whose protein product is easily assayed may be used for convenient detection. Reporter genes include, for example, alkaline phosphatase, β-galactosidase (LacZ), chloramphenicol acetyltransferase (CAT), α-glucoronidase (GUS), bacterial/insect/marine invertebrate luciferases (LUC), green and red fluorescent proteins (GFP and RFP, respectively), horseradish peroxidase (HRP), β-lactamase, and derivatives thereof (e.g., blue EBFP, cyan ECFP, yellow-green EYFP, destabilized GFP variants, stabilized GFP variants, or fusion variants sold as LIVING COLORS fluorescent proteins by Clontech). Reporter genes would use cognate substrates that are preferably assayed by a chromogen, fluorescent, or luminescent signal. Alternatively, assay product may be tagged with a heterologous epitope (e.g., FLAG, MYC, SV40 T antigen, glutathione transferase, hexahistidine, maltose binding protein) for which cognate antibodies or affinity resins are available. Examples of drugs for which selectable marker genes exist are ampicillin, geneticin/kanamycin/neomycin, hygromycin, puromycin, and tetracycline. A metabolic enzyme (e.g., dihydrofolate reductase, HSV-1 thymidine kinase) may be used as a selectable marker in sensitive host cells or auxotrophs. For example, methotrexate can increase the copy number of a polynucleotide linked to a DHFR selectable marker and gancyclovir can negatively select for a viral thymidine kinase selectable marker.

A polynucleotide may be ligated to a linker oligonucleotide or conjugated to one member of a specific binding pair (e.g., antibody-digoxygenin/hapten/peptide epitope, biotin-avidin/streptavidin, glutathione S transferase or GST-glutathione, lectin-sugar, maltose binding protein-maltose, polyhistidine-nickel, protein A/G-immunoglobulin). The polynucleotide may be conjugated by ligation of a nucleotide sequence encoding the binding member. A polypeptide may be joined to one member of the specific binding pair by producing the fusion encoded by such a ligated or conjugated polynucleotide or, alternatively, by direct chemical linkage to a reactive moiety on the binding member by chemical cross-linking. Such polynucleotides and polypeptides may be used as an affinity reagent to identify, to isolate, and to detect interactions that involve specific binding of a transcript or protein product of the expression vector. Before or after affinity binding of the transcript or protein product, the member attached to the polynucleotide or polypeptide may be bound to its cognate binding member. This can produce a complex in solution or immobilized to a support. A protease recognition site (e.g., for enterokinase, Factor Xa, ICE, secretases, thrombin) may be included between adjoining domains to permit site specific proteolysis that separates those domains and/or inactivates protein activity.

Probes and primers may be used to identify a fungal species or variant thereof. For example, a probe or primer specific for one of the three bot1 genes identified herein may be used to detect the presence or absence of the gene, and thereby infer that the fungal source of the gene is present or absent, respectively. Genetic polymorphisms and mutations in the bot1 gene may be specifically detected by positioning a potentially mismatched base(s) in the middle portion of a probe or the 3′-end of a primer to stabilize or to destabilize binding of the probe or primer to its target depending on whether the target's sequence at that position is complementary to the base or not, respectively.

Genetic polymorphisms and mutations may also be detected by a change in the length of a restriction fragment (RFLP), nuclease-protected fragment (e.g., S1 nuclease, deoxyribonuclease 1, ribonuclease A), or amplified product. For complicated genetic fingerprints, identification of each of the component of the fingerprint may not be needed because a comparison might easily detect differences (e.g., RAPD). Differences may also be detected by changes in the molecular weight (Mw) or isoelectric point (pl) of the Bot1 protein by gel electrophoresis or isoelectric focusing, respectively.

For example, Candida strains or species may be distinguished by differences in the bot1 gene and/or Bot1 protein. In this manner, drug-sensitive and/or drug-resistant fungi may be identified, molecular phylogeny may be analyzed, infections may be tracked in a population, routes of contamination may be determined, or combinations thereof. A fungal infection caused by Candida or Aspergillus may be distinguished because the bot1 gene and/or Bot1 protein in Candida is not detectable in Aspergillus. This may allow phylogenetically distinguishing between yeasts and filamentous fungi.

Presence of Bot1 nucleic acid or protein may be used as a marker for infection in human or animal fluids or tissues. The fluid may be blood, blood product (e.g., plasma, serum), cerebrospinal fluid, lavage, sputum, or the like. Exemplary tissues are those of the epithelium (e.g., lung) or mucosa (e.g., mouth, vagina), although infection may be systemic and involve other tissue types as well. Signal may be detected in situ for solid tissue, on dispersed or homogenized tissue, in solution (e.g., diluted or undiluted body fluid, wash), or on a cell smear or touch prep.

Construction of Expression Vector

An expression vector is a recombinant polynucleotide that is in chemical form either a deoxyribonucleic acid (DNA) and/or a ribonucleic acid (RNA). The physical form of the expression vector may also vary in strandedness (e.g., single-stranded or double-stranded) and topology (e.g., linear or circular). The expression vector is preferably a double-stranded deoxyribonucleic acid (dsDNA) or is converted into a dsDNA after introduction into a cell (e.g., insertion of a retrovirus into a host genome as a provirus). The expression vector may include one or more regions from a mammalian, insect, plant or fungal gene or a virus (e.g., adenovirus, adenoassociated virus, cytomegalovirus, fowlpox virus, herpes simplex virus, lentivirus, Moloney leukemia virus, mouse mammary tumor virus, Rous sarcoma virus, SV40 virus, vaccinia virus), as well as regions suitable for genetic manipulation (e.g., selectable marker, linker with multiple recognition sites for restriction endonucleases, promoter for in vitro transcription, primer annealing sites for in vitro replication).

The expression vector may be associated with proteins and other nucleic acids in a carrier (e.g., packaged in a viral particle) or condensed with a chemical (e.g., cationic polymer) to target entry into a cell or tissue. The expression vector further comprises a regulatory region for gene expression (e.g., promoter, enhancer, silencer, splice donor or acceptor site, polyadenylation signal, cellular localization sequence). Different levels of transcription can be achieved using an agent (e.g., antibiotic or vitamin) with a regulatory system which responds to the agent (e.g., tetracycline/tetR or thiamine/nmt1, respectively). The expression vector may be further comprised of one or more splice donor and acceptor sites within an expressed region; Kozak consensus sequence upstream of an expressed region for initiation of translation; and downstream of an expressed region, multiple stop codons in the three forward reading frames to ensure termination of translation, one or more mRNA degradation signals, a termination of transcription signal, a polyadenylation signal, and a 3′ cleavage signal. For expressed regions that do not contain an intron (e.g., a coding region from a cDNA), a pair of splice donor and acceptor sites may or may not be preferred. It would be useful, however, to include mRNA degradation signal(s) if it is desired to express one or more of the downstream regions only under the inducing condition.

An origin of replication (ARS) may also be included that allows replication of the expression vector integrated in the host genome or as an autonomously replicating episome. Centromere and telomere sequences can also be included for the purposes of chromosomal segregation and protecting chromosome ends, respectively. Random or targeted integration into the host genome is more likely to ensure maintenance of the expression vector but episomes could be maintained by selective pressure or, alternatively, may be preferred for those applications in which the expression vector is present only transiently.

An expressed region may be derived from any gene of interest, and provided in either orientation with respect to the promoter; the expressed region in the anti-sense orientation will be useful for making cRNA and antisense polynucleotide. The gene may be derived from the host cell or organism, from the same species thereof, or designed de novo; but it is preferably of archael, bacterial, fungal, plant, or animal origin. Fusions with a domain(s) of genes that may share a function with Bot1 (e.g., Mob2, Mor2, Orb6, Orb11, Skb1, Tea1) can be assayed to define the domain(s) that confers the function or to provide a multifunctional fusion protein. A fusion may also be made with an epitope tag (e.g., GFP, GST, HA, MYC, TAP). Some genes produce alternative transcripts, encode subunits that are assembled as homopolymers or heteropolymers, or produce propeptides that are activated by protease cleavage. The expressed region may encode a translational fusion; open reading frames of the regions encoding a polypeptide and at least one heterologous domain may be ligated in register. If a reporter or selectable marker is used as the heterologous domain, then expression of the fusion protein may be readily assayed or localized. The heterologous domain may be an affinity or epitope tag.

Genetic manipulation of fungi may be performed using materials and techniques known in the art. See, for example, Alfa et al. (Experiments with Fission Yeast, CSHL Press, 1993); Burke et al. (Methods in Yeast Genetics, CSHL Press, 2000); Celis (Cell Biology, A Laboratory Manual, 2^{nd} Ed., Academic Press, 1997); Guthrie et al., (Guide to Yeast Genetics and Molecular and Cell Biology, Methods in Enzymology, vols. 194, 1991 and 350-351, 2002); Wheals et al., (Yeast Genetics, The Yeasts, vol. 6, 1995)<URL:http://alces.med.umn.edu/Candida.html>; <URL:http://www.bio.uva.nl/pombe/>; <URL:http://biosci.cbs.umn.edu/labs/berman/index.htm>.

Screening of Candidate Compounds

Another aspect of the invention are chemical or genetic compounds, derivatives thereof, and compositions including same that are effective in treatment of fungal disease and individuals at risk thereof. The amount that is administered to an individual in need of therapy or prophylaxis, its formulation, and the timing and route of delivery is effective to reduce the number or severity of symptoms, to slow or limit progression of symptoms, to inhibit expression of one or more genes that are transcribed during an infection, to activate expression of one or more genes that are transcribed at a lower level in an infection, or any combination thereof. Determination of such amounts, formulations, and timing and route of drug delivery is within the skill of persons conducting in vitro assays of Bot1 protein activity, in vivo studies of animals infected by fungus, and human clinical trials.

A screening method may comprise administering a candidate compound to an organism or incubating a candidate compound with a cell, and then determining whether or not gene expression is modulated. Such modulation may be an increase or decrease in activity that partially or fully compensates for a change that is associated with or may cause symptoms of fungal disease. Gene expression may be increased at the level of rate of transcriptional initiation, rate of transcriptional elongation, stability of transcript, translation of transcript, rate of translational initiation, rate of translational elongation, stability of protein, rate of protein folding, proportion of protein in active conformation, functional efficiency of protein (e.g., activation or repression of transcription), or combinations thereof. See, for example, U.S. Pat. Nos. 5,071,773 and 5,262,300. High-throughput screening assays are possible (e.g., by using parallel processing and/or robotics).

The screening method may comprise incubating a candidate compound with a cell containing a reporter construct, the reporter construct comprising a transcriptional regulatory region of bot1 covalently linked in a cis configuration to a downstream gene encoding an assayable product; and measuring production of the assayable product. Either a chimera with an upstream region of the bot1 gene (e.g., about 200 bases to about 2000 bases) or a translational fusion in frame with the initiating ATG codon may be used to provide the transcriptional regulatory region. A candidate compound which increases production of the assayable product would be identified as an agent which activates gene expression while a candidate compound which decreases production of the assayable product would be identified as an agent which inhibits gene expression. See, for example, U.S. Pat. Nos. 5,849,493 and 5,863,733.

The screening method may comprise measuring in vitro transcription from a reporter construct in the presence or absence of a candidate compound (the reporter construct comprising a transcription regulatory region) and then determining whether transcription is altered by the presence of the candidate compound. In vitro transcription may be assayed using a cell-free extract, partially purified fractions of the cell, purified transcription factors or RNA polymerase, or combinations thereof. See, for example, U.S. Pat. Nos. 5,453,362; 5,534,410; 5,563,036; 5,637,686; 5,708,158; and 5,710,025.

Techniques for measuring transcriptional or translational activity in vivo are known in the art. For example, a nuclear run-on assay may be employed to measure transcription of a reporter gene. Translation of the reporter gene may be measured by determining the activity of the translation product. The activity of a reporter gene can be measured by determining one or more of transcription of polynucleotide product (e.g., RT-PCR of GFP transcripts), translation of polypeptide product (e.g., immunoassay of GFP protein), and enzymatic activity of the reporter protein per se (e.g., fluorescence of GFP or energy transfer thereof).

A compound that reduces the expression or activity of an essential gene could then be assayed for its effect on slowing growth of a fungus, decreasing the viability of the fungus, reducing the likelihood or severity of infection in an individual, abundance or biological activity of a Bot1-containing membrane-associated complex, improving the mortality or morbidity of disease, or combinations thereof.

Abundance, assembly, biological activity, or combinations thereof of a Bot1-containing membrane-associated complex may also be assayed. An epitope-tagged Bot1 protein or antibody specific for Bot1 protein may be used to affinity purify the complex. Candidate compounds may be screened for their ability to decrease the abundance (i.e., steady-state level of complex), rate of assembly, or biological activity of the complex. Other components of the complex (e.g., Tea1 and Orb6) may be identified by screening for synthetic lethal mutations or observing their downregulation in a Bot1-deficient fungus. For example, a comparison of protein patterns (e.g., two-dimensional SDS-polyacrylamide gel electrophoresis and isoelectric focusing) between a first fungus with a functional Bot1 protein and a second fungal mutant which has been deleted for the bot1 gene or in which bot1 gene expression has been reduced may show certain protein is co-regulated with Bot1 protein, and may be suspected thereby of being other components of the complex. Examples of using in vitro or bacterially expressed proteins and two- or three-hybrid interaction screens to identify other components that participate in complex formation are shown below. Similarly, a protein or other compound which inhibits binding between Bot1 protein and another component of the complex may be identified.

Candidate compounds regulating the binding between Bot1 protein and other components of the complex (e.g., Tea1 and Orb6) may be identified. Bot1 protein can be attached to a substrate as described above. A candidate compound is incubated with the immobilized Bot1p protein in the presence of at least one other component of the complex in at least partially purified form or as a crude mixture. Moreover, one or more components of the complex can be attached to a substrate and a candidate compound can be incubated with the immobilized component in the presence of Bot1 protein with or without additional components of the complex in at least partially purified form or as a crude mixture. Examples of conditions for binding are shown below. After incubation, all nonbinding components can be washed away, leaving one or more components of the complex bound to the substrate. Complex formation including Bot1 protein may also take place in solution and then the Bot1-containing complex may be immobilized or not. The amount of each component of the complex can then be quantified after washing and separation of the complex from other proteins (e.g., heterogeneous assay) or without separation (e.g., homogeneous assay). For example, it can be determined using an immunological assay, such as ELISA, RIA, or Western blotting. Complex formation may be determined by binding of an antibody to an epitope which is dependent on formation or an epitope which is masked after formation. Complex may be immobilized before or after formation by binding at least one component of the complex to a substrate. Binding of complex to a substrate may be determined without separation by proximity detection, such as SPA or BiaCore. The amount of the one or more bound components of the complex is determined with and without the candidate compound. A desirable compound is one which decreases the abundance, assembly, biological activity, or combinations thereof of a Bot1-containing membrane-associated complex.

One or two alleles of the bot1 gene may be put under control of an inducible promoter in a fungus. It may be used to assess the hypersensitivity to compounds when Bot1 is underexpressed or the hyposensitivity to compounds when over-expressed. Any fungus may be used as a source of the bot1 gene or as the host for the mutant (e.g., C. albicans, S. pombe, S. cerevisiae, and mutant strains thereof). The gene and host may represent the same or different fungal species. If Bot1 is expressed in a heterologous fungus (e.g., bot1 gene from C. albicans inserted into a S. pombe host under the control of the nmt1 promoter), the endogenous bot1 gene may be deleted. If overexpression of the bot1 gene is toxic in a heterologous host, candidate compounds may be screened for those that inhibit toxicity because interfering with binding of Bot1 protein to endogenous host factors or other components of the complex may provide an antifungal agent.

Genetic Compounds for Treatment

Gene activation may be achieved by inducing an expression vector containing a downstream region related to a gene that is down regulated (e.g., the full-length coding region or functional portions of the gene; hypermorphic mutants, homologs, orthologs, or paralogs thereof) or unrelated to the gene that acts to relieve suppression of gene activation (e.g., at least partially inhibiting expression of a negative regulator of the gene). Overexpression of transcription or translation, as well as overexpressing protein function, is a more direct approach to gene activation. Alternatively, the downstream expressed region may direct homologous recombination into a locus in the genome and thereby replace an endogenous transcriptional regulatory region of the gene with an expression cassette.

An expression vector may be introduced into a host mammalian, insect, plant, or fungal cell or tissue, or nonhuman mammal, insect, plant, or fungus by a transfection or transgenesis technique using, for example, one or more chemicals (e.g., calcium phosphate, DEAE-dextran, lipids, polymers), biolistics, electroporation, naked DNA technology, microinjection, or viral infection. The introduced expression vector may integrate into the host genome of the cell or whole organism, or be maintained as an episome. Many neutral and charged lipids, sterols, and other phospholipids to make lipid carriers are known. For example, neutral lipids are dioleoyl phosphatidylcholine (DOPC) and dioleoyl phosphatidyl ethanolamine (DOPE); an anionic lipid is dioleoyl phosphatidyl serine (DOPS); cationic lipids are dioleoyl trimethyl ammonium propane (DOTAP), dioctadecyldiamidoglycyl spermine (DOGS), dioleoyl trimethyl ammonium (DOTMA), and 1,3-dioleoyloxy-2-(6-carboxyspermyl)-propylamide tetraacetate (DOSPER). Dipalmitoyl phosphatidylcholine (DPPC) can be incorporated to improve the efficacy and/or stability of delivery. FUGENE 6, LIPOFECTAMINE, LIPOFECTIN, DMRIE-C, TRANSFECTAM, CELLFECTIN, PFX-1, PFX-2, PFX-3, PFX-4, PFX-5, PFX-6, PFX-7, PFX-8, TRANSFAST, TFX-10, TFX-20, TFX-50, and LIPOTAXI lipids are proprietary formulations. The polymer may be cationic dendrimer, polyamide, polyamidoamine, polyethylene or polypropylene glycol (PEG), polyethylenimine (PEI), polylysine, or combinations thereof; alternatively, polymeric material can be formed into nanoparticle or microparticle. In naked DNA technology, the expression vector (usually as a plasmid) is delivered to a cell or tissue, where it may or may not become integrated into the host genome, without using chemical transfecting agents (e.g., lipids, polymers) to condense the expression vector prior to its introduction into the cell or tissue.

A mammalian, insect, plant or fungal cell may be transfected; also provided is a transgenic nonhuman mammal, insect, plant or fungus. In the previously discussed alternative, a homologous region from a gene can be used to direct integration to a particular genetic locus in the host genome and thereby regulate expression of the gene at that locus (e.g., homologous recombination of a promoterless reporter or selectable marker at the bot1 genetic locus) or ectopic copies of the bot1 gene may be inserted. Polypeptide may be produced in vitro with a cell extract or in vivo with a genetically manipulated cell, The expression vector may be used to replace function of a gene that is down regulated or totally defective, supplement function of a partially defective gene, or compete with activity of the gene. Thus, the cognate gene activity of the host may be neomorphic, hypomorphic, hypermorphic, or normal. Replacement or supplementation of function can be accomplished by the methods discussed above, and the genetically manipulated fungus may be selected for high or low expression (e.g., assessing the amount of transcribed or translated produce, or the physiological function of either product) of the downstream region. But competition between the expressed downstream region and a neomorphic, hypermorphic, or normal gene may be more difficult to achieve unless the encoded polypeptides are multiple subunits that form into a polymeric protein complex (e.g., synthetic interaction). Alternatively, a negative regulator or a single-chain antibody that inhibits function intracellularly may be encoded by the downstream region of the expression vector. Therefore, at least partial inhibition of genes that are required for fungal disease may use antisense, ribozyme, or triple helix technology in which the expression vector contains a downstream region corresponding to the unmodified antisense molecule, ribozyme, or triple helix molecule, respectively.

Antisense polynucleotides were initially believed to directly block translation by hybridizing to mRNA transcripts, but may involve degradation of such transcripts of a gene. The antisense molecule may be recombinantly made using at least one functional portion of a gene in the antisense orientation as a downstream expressed region in an expression vector. Chemically modified bases or linkages may be used to stabilize the antisense polynucleotide by reducing degradation or increasing half-life in the body (e.g., methyl phosphonates, phosphorothIoate, peptide nucleic acids). The sequence of the antisense molecule may be complementary to the translation initiation site (e.g., between −10 and +10 of the target's nucleotide sequence).

Ribozymes catalyze specific cleavage of an RNA transcript or genome. The mechanism of action involves sequence-specific hybridization to complementary cellular or viral RNA, followed by endonucleolytic cleavage. Inhibition may or may not be dependent on ribonuclease H activity. The ribozyme includes one or more sequences complementary to the target RNA as well as catalytic sequences responsible for RNA cleavage (e.g., hammerhead, hairpin, axehead motifs). For example, potential ribozyme cleavage sites within a subject RNA are initially identified by scanning the subject RNA for ribozyme cleavage sites which include the following trinucleotide sequences: GUA, GUU and GUC. Once identified, an oligonucleotide of between about 15 and about 20 ribonucleotides corresponding to the region of the subject RNA containing the cleavage site can be evaluated for predicted structural features, such as secondary structure, that can render candidate oligonucleotide sequences unsuitable. The suitability of candidate sequences can then be evaluated by their ability to hybridize and cleave target RNA.

Molecules used in triplex helix formation for inhibiting expression of a gene that is up regulated should be single-stranded and composed of deoxyribonucleotides. The base composition of these oligonucleotides must be designed to promote triple helix formation by Hoogsteen base pairing rules, which generally require sizeable stretches of either purines or pyrimidines to be present on one strand of the duplex. Nucleotide sequences can be pyrimidine-based and result in TAT and CGC triplets across the three associated strands. The pyrimidine-rich molecules provide base complementarity to a purine-rich region of a single strand of the duplex in a parallel orientation to that strand. In addition, triple helix forming molecules can be chosen that are purine-rich (e.g., containing a stretch of guanines). These molecules may form a triple helix with a DNA duplex that is rich in GC pairs, in which the majority of the purines are located on a single strand of the targeted duplex, resulting in GGC triplets across the three strands in the triplex.

Antibody specific for Bot1 or a gene product increased during infection can be used for inhibition or detection. Polyclonal or monoclonal antibodies may be prepared by immunizing animals (e.g., chicken, hamster, mouse, rat, rabbit, goat, horse) with antigen, and optionally affinity purified against the same or a related antigen. Antigen may be native protein, fragment made by proteolysis or genetic engineering, fusion protein, or in vitro translated or synthesized protein which includes at least one or more epitopes bound by the antibody. Antibody fragments may be prepared by proteolytic cleavage or genetic engineering; humanized antibody and single-chain antibody may be prepared by transplanting sequences from antigen binding domains of an antibody to framework molecules. In general, other binding molecules may be prepared by screening a combinatorial library for a member which specifically binds antigen (e.g., phage display library). Antigen may be a full-length protein encoded by the gene or fragment(s) thereof. The antibody may be specific for Bot1 from a limited number of fungal species or it may cross react against a broad range of fungal species depending on how well the epitope recognized by the antibody is conserved among different species. See, for example, U.S. Pat. Nos. 5,403,484; 5,723,286; 5,733,743; 5,747,334; and 5,871,974.

Bot1-specific binding agents (e.g., polynucleotides, polypeptides) may be used diagnostically to detect Bot1 nucleic acid or protein, or for treatment to inhibit Bot1 activity (e.g., transcription, translation, cellular localization, enzymology, viability).

Formulation of Compositions

Compounds of the invention or derivatives thereof may be used as a medicament or used to formulate a pharmaceutical composition with one or more of the utilities disclosed herein. They may be administered in vitro to cells in culture, in vivo to cells in the body, or ex vivo to cells outside of an individual which may then be returned to the body of the same individual or another. Such cells may be disaggregated or provided as solid tissue.

Compounds or derivatives thereof may be used to produce a medicament or other pharmaceutical compositions. Use of compositions which further comprise a pharmaceutically acceptable carrier and compositions which further comprise components useful for delivering the composition to an individual are known in the art. Addition of such carriers and other components to the composition of the invention is well within the level of skill in this art.

Pharmaceutical compositions may be administered as a formulation adapted for passage through the gut or blood circulation. Alternatively, pharmaceutical compositions may be added to the culture medium. In addition to active compound, such compositions may contain pharmaceutically-acceptable carriers and other ingredients known to facilitate administration and/or enhance uptake (e.g., saline, dimethyl sulfoxide, lipid, polymer, affinity-based cell specific-targeting systems). The composition may be administered in a single dose or in multiple doses which are administered at different times.

Pharmaceutical compositions may be administered by any known route. By way of example, the composition may be administered by a mucosal, pulmonary, topical, or other localized or systemic route (e.g., enteral and parenteral). The term “parenteral” includes subcutaneous, intradermal, intramuscular, intravenous, intraarterial, intrathecal, and other injection or infusion techniques, without limitation.

Suitable choices in amounts and timing of doses, formulation, and routes of administration can be made with the goals of achieving a favorable response in the individual with fungal disease or at risk thereof (i.e., efficacy), and avoiding undue toxicity or other harm thereto (i.e., safety). Therefore, “effective” refers to such choices that involve routine manipulation of conditions to achieve a desired effect.

A bolus of the formulation administered to an individual over a short time once a day is a convenient dosing schedule. Alternatively, the effective daily dose may be divided into multiple doses for purposes of administration, for example, two to twelve doses per day. Dosage levels of active ingredients in a pharmaceutical composition can also be varied so as to achieve a transient or sustained concentration of the compound or derivative thereof in an individual and to result in the desired therapeutic response or protection. But it is also within the skill of the art to start doses at levels lower than required to achieve the desired therapeutic effect and to gradually increase the dosage until the desired effect is achieved.

The amount of compound administered is dependent upon factors known to a person skilled in the art such as bioactivity and bioavailability of the compound (e.g., half-life in the body, stability, and metabolism); chemical properties of the compound (e.g., molecular weight, hydrophobicity, and solubility); route and scheduling of administration; and the like. It will also be understood that the specific dose level to be achieved for any particular individual may depend on a variety of factors, including age, gender, health, medical history, weight, combination with one or more other drugs, and severity of disease.

The term “treatment” of fungal disease refers to, inter alia, reducing or alleviating one or more symptoms in an infected individual, preventing one or more symptoms from worsening or progressing, promoting recovery or improving prognosis, preventing disease, or combinations thereof in an individual who is free therefrom as well as slowing or reducing progression of existing disease. For a given individual, improvement in a symptom, its worsening, regression, or progression may be determined by an objective or subjective measure. Efficacy of treatment may be measured as an improvement in morbidity or mortality (e.g., lengthening of survival curve for a selected population). Prophylactic methods (e.g., preventing or reducing the incidence of relapse) are also considered treatment. Treatment may also involve combination with other existing modes of treatment and antifungal agents. Thus, combination treatment with one or more other drugs and one or more other medical procedures may be practiced.

### EXAMPLES

The microtubule cytoskeleton, which is essential for vesicle transport to the cell surface (58), is fundamental to cell morphogenesis (19). For example, in epithelial cells, long stable bundles of microtubules run along the apical-basal axis of the cell with their plus ends towards the basal end, while in neuronal axons, microtubules are organized longitudinally with their plus ends towards the axon extremity. Disruption of the microtubule cytoskeleton leads to delocalization of components of the plasma membrane in epithelial cells and to neurite retraction in neurons. The process of spatial localization of signaling molecules shows how microtubules may contribute to the spatial organization of the actin cytoskeleton (19).

Fission yeast Schizosaccharomyces pombe is an excellent model system for studies of cell morphogenesis because it grows in a polarized fashion with a well-defined cylindrical shape. Generation of cell shape in fission yeast is tightly coordinated with the cell cycle. Following cytokinesis, the newly divided daughter cells initiate growth in a monopolar fashion from the cell tip that was growing in the previous cell cycle (Old End Take Off) (55). In early G2, after the attainment of a critical cell length, cells switch to a bipolar growth pattern by activating the second end (New End Take Off or NETO) (45). Bipolar growth continues through ˜0.75 of the cell cycle when tip elongation ceases and mitosis occurs.

Interestingly, disruption of the microtubule cytoskeleton in fission yeast induces cell distortion and cell branching, indicating an important role for the microtubules in cell morphology (22) and that S. pombe is a valuable model system for studying control of microtubule-dependent cell polarity. Nineteen fission yeast genes important for various aspects of cell morphogenesis have been identified, and classified them according to their functions during the cell cycle (61). One of these, called tea1, encodes a protein that localizes to the cell tips in a microtubule-dependent fashion and functions as a molecular marker for the correct placement of the growth sites (40, 57, 61). Following association with the cell tips, Tea1p presumably controls polarity of cell growth by binding to a number of downstream effectors. Consistent with this hypothesis, Tea1p has been found to be a component of a large protein complex (18, 40). The mechanism of assembly of the Tea1p complex and its protein composition has been poorly understood. Thus, the identification of its components will be fundamental to understanding Tea1p function in cell polarity and of microtubule-dependent growth control.

Another gene orb6 is required for maintenance of cell polarity, for polarized localization of the actin cytoskeleton, and for its reorganization during the cell cycle (62). Orb6 encodes a conserved protein kinase related to human, C. elegans, and Drosophila Ndr kinases (44), S. cerevisiae Cbk1p (51), and Neurospora Cot1 (68). These kinases are related to mammalian Rho-kinase (30, 33, 41) but lack the consensus Rho-binding motifs. Cot1 and Cbk1p have also been shown to be required for the regulation of cell morphology (5, 51, 68). Similarly, C. elegans Ndr/Sax-1 kinase is involved in the control of neuronal cell shape and neurite outgrowth (69). The mechanisms of regulation and the role played by these kinases in cell polarity are still poorly understood.

To explore the function of Orb6p in the control of cell morphogenesis, a two-hybrid screen was performed to identify proteins that interact with Orb6p. Here, one of these factors, Bot1p, is characterized. Bot1p is essential for cell viability, normal cell morphogenesis, and proper Orb6p kinase localization. Interestingly, we found that Bot1p associates with Tea1p in the two-hybrid system, in cell extracts and in vitro, and that it is dependent on Tea1p for its normal localization. These results indicate that Tea1p has a role in recruiting Bot1p to the cell tip. Bot1p then controls the proper localization of components essential for polarized cell growth, and specifically of Orb6p. These findings provide a first characterization of a Tea1-associated protein and a Tea1 effector in the control of polarized cell growth, and suggest a mechanism by which the microtubule cytoskeleton contributes to the spatial localization of signaling molecules.

Strains and Cell Culture

Schizosaccharomyces pombe strains used here included the wild-type 972 cells and mutant strains tea1-1 leu1-32 ade6-M210 h-, tea1::ura4+ ura4-D18 leu1-32 h-, and orb6::ura4+ ura4-D18 ade-leu1-32 h90. All fission yeast strains used were isogenic to 972 (see Table of Strains). Cells were cultured in YE (yeast extract rich medium) or MIN medium (46) with the appropriate supplements at the indicated temperatures. Genetic manipulations and analysis were performed by standard procedures (40).

Saccharomyces cerevisiae strain Y190 (MATa gal4 gal80 his3 trp1-901 ade2-101 ura3-52 leu2-3,-112, URA3::GAL-lacZ, LYS2::GAL(UAS)-HIS3 cyh^{r}) was used as the host for the two-hybrid interaction experiments. Strain Y187 (MATaga/4 gal80 his3 trp1-901 ade2-101 ura3-52 leu2-3,-112 met-URA3::GAL-lacZ) was used for mating experiments (14). Strain BY3161 (MATa leu2-3 trp1-901 his3-200 ura3-52 ade2-101 gal4-542 gal80-538 GAL1-lacZ GAL1-His3) was used for three-hybrid interaction studies. Cells were cultured in YAPD or selective SC at 30° C. Three-aminotriazole (3-AT) was added to the plates when selecting for histidine prototrophy.

Two-Hybrid Screen for Proteins Interacting With Tea1 and Orb6

The tea1 gene was fused to the DNA binding domain of GAL4 in a plasmid (pAS1) carrying the trp1 marker. The orb6 gene was fused to the DNA binding domain of GAL4 (pAS1-orb6) and to the GAL4 activator domain (pACT2-orb6). The cDNA library was cloned to the transcription activator domain of GAL4 in a plasmid (pACT) carrying the leu2 marker. Plasmids pAS1, pACT2, pSE1111 (pACT-SNF4), pSE1112 (pAS1-SNF1), pAS1-p53, pAS1-lamin and the S. pombe library prepared in phage λ vectors and plasmids pAS1-SNF1 and pAS1-lamin, both harbored in Y187 strain, were kindly provided by Dr. S. Elledge. The test for cell growth on medium lacking histidine, the beta-galactosidase activation assay and the mating test to detect interaction specificity were performed as described (14). After screening 2×10⁶ transformants, we found five proteins that interact with Orb6p and four that interact with Tea1p in this system, and that were identified multiple times in the screen. Clones containing bot1+ were isolated 22 times in the Orb6p screen and eight times in the Tea1p screen. All clones contained the full-length bot1 sequence, although they usually lacked the initial ATG codon. For beta-galactosidase activation, three independent transformants were assayed for each condition.

We also investigated whether Bot1p had been isolated in other screens using the same reagents, but different baits. By dot blotting, it was confirmed that Bot1p was not identified in screens using fission yeast Cdt1p, Cdc23p or Sds23p as bait. The pACT plasmids isolated through screens using pAS1-cdt1, pAS1-cdc23 and pAS1-sds23, were kindly provided by W. Feng and Dr. G. D'Urso (University of Miami). Cdc23p is involved in the control of DNA replication (3), Cdt1p in cell cycle control (28), and Sds23p in the regulation of APC function (29). Two-hybrid screens of 2×10⁶ transformants using Cdc23p, Cdt1p or Sds23p as bait identified 24, 80 and 25 positive clones, respectively.

To test for a ternary complex between Tea1p, Orb6p and Bot1p, pACT2-tea1 and pAS1-orb6 were transformed into BY3161 along with the bot1 gene on a yeast plasmid carrying the Ura3+ marker (pDela bot1+). An empty Ura3⁺ plasmid (pDela; 70) was used as a control.

Construction of the Bot1 Deletion Strain

Deletion of bot1+ was performed by substituting the whole bot1 ORF (residues 1 to 316) with the ura4+ sequence. The 3′ and 5′ flanking sequences were obtained by PCR from the bot1 containing c14C8 (Sanger Center, UK) cosmid. After transformation, nine ura4+ diploids were analyzed for the presence of the bot1 deletion by Southern blotting. Eight were found to contain the deletion. Two diploids were chosen, sporulated and analyzed by tetrad analysis. Ten complete tetrads showed 2:2 segregation of the deletion phenotype, which was lethal. The viable spores produced ura4-colonies and wild-type-looking cells. The deleted spores produced nonviable microcolonies with morphologically aberrant cells.

To test the effects of lowered levels of Bot1p, a bot1::ura4+/bot1+ ade6-M210/ade6-M216 ura4-D18/ura4-D18 leu1-32/leu1-32 h+/h-diploid was transformed with the integrative plasmid pJK148 containing the gene bot1+ under the control of the nmt1 promoter; the diploid was then sporulated and haploid ura+ leu+ colonies were selected. In medium lacking thiamine, expression of bot1+ from a full-strength nmt1 promoter fully rescued the lethality and phenotype of the bot1 deletion. When integrated bot1 was expressed from a more attenuated nmt1 promoter, the resulting strain displayed a very slow growing phenotype and misshapen, bottle-shaped cells. These results demonstrate a dose-dependent relationship between Bot1p activity and function.

Expression of Orb6p-HA Under the Control of the Endogenous orb6 Promoter

One promoter-less copy of the orb6+ gene, tagged at the C terminus with three copies of the HA epitope, was inserted in an integrative plasmid containing the sup3-5 marker. The plasmid was then transformed in ade6-704 leu1-32 ura4-D18 h-cells and selected for colonies capable to grow in the absence of adenine. PCR with a flanking primer confirmed integration at the correct locus and that the HA-tagged orb6+ gene was correctly positioned after the endogenous orb6 promoter. Three strains were used to produce cell extracts and to test on Western blots for the expression of a protein corresponding to the size of Orb6p-HA. In all cases, a single band of the expected size was recognized by the HA antibody (Covance).

Immunofluorescence Microscopy

Cells in liquid cultures were grown exponentially for at least eight generations, at densities below 10⁷ cells/ml, before the start of the experiment. Immunofluorescent staining was performed as described (46). Cells were fixed in methanol and stained with the following primary antibodies: a rabbit polyclonal antibody anti-Tea1p (40, a kind gift of Dr. P. Nurse, ICRF London), a rabbit polyclonal antibody anti-Orb6p, a monoclonal anti-hemagglutinin (HA) antibody (Covance), and a chicken polyclonal anti-Myc antibody (Molecular Probes). For actin and microtubule staining, a monoclonal anti-actin antibody (Amersham Pharmacia Biotech) and a monoclonal anti-tubulin antibody (TAT1; a kind gift of Dr. K. Gull), respectively, were used A CY3-conjugated anti-mouse, a CY3-conjugated anti-rabbit, or an FITC-conjugated anti-mouse were used as secondary antibodies (Sigma). Cells were immobilized on coverslips using PBS-containing antifade (Molecular Probes) as mounting medium and photographed using a Zeiss Axiophot microscope or a Leica DMRA microscope, equipped with Metamorph 1.7.4 software (Universal Images).

Co-Purification and Immunoblot Analysis

Immunoblot analysis was performed using standard methodology. For co-purification experiments, ade6-704 ura4-D18 leu1-32 h-cells co-expressing HA-tagged Bot1p and GST-tagged Orb6p (FV17p), or cells co-expressing HA-tagged Bot1p and GST alone (FV16p) as a control were cultured, for 15 hr at 32° C. in the absence of thiamine. HA₃-bot1 was moderately expressed from an attenuated nmt1 promoter (10). GST-tagged fusions were constructed using the pESP plasmid (Qiagen), which also contains a nmt1 promoter. Cells were then re-suspended in HB buffer and broken using a FastPrep FP120 bead beater (Savant). Extracts were spun at 15K rpm for 15 min. GST-tagged Orb6 or GST alone was purified using glutathione beads (Molecular Probes).

When co-purification of GST-Tea1p and HA-Bot1p was attempted, a different procedure was used because co-expression was found to be detrimental to cell growth. ade6-704 ura4-D18 leu1-32 h-cells expressing GST-tagged Tea1p (FV307p), cells expressing GST alone (FV308p), cells expressing HA-Bot1p (FV370p) or cells expressing HA-Cdc23 (FV372p) were cultured for 15 hr at 32° C. in the absence of thiamine. Cell extracts were prepared as described. GST-Tea1p and GST were purified using glutathione beads (Molecular Probes). Beads were spun, and further incubated for 4 hr with an extract containing HA-Bot1p or a control extract containing HA-Cdc23p.

Glutathione beads were washed three times in HB buffer and then eluted. The eluate was run on a 10% polyacrylamide gel, blotted on nitrocellulose, and probed with a primary anti-HA antibody (Covance) or, separately, with a primary anti-GST antibody (Molecular Probes) and with a horseradish peroxidase-conjugated secondary antibody (ECL, Amersham).

In Vitro Assay for Interactions Between Bot1p, Tea1p and Orb6p

To test for in vitro interaction between Bot1p and Tea1p, the N-terminal domain of Tea1p was fused to a His tag (pQE30-tea1[10-553]). His-tagged Tea1p was expressed in bacteria, and then bound to Ni²⁺-NTA magnetic agarose beads (Qiagen). The beads were then washed with a phosphate buffer (50 mM NaH₂PO₄, 500 mM NaCl, 40 mM imidazole, 0.2% BSA, 0.005% Tween-20, pH 8.0). ³⁵S-labeled Bot1p was expressed from plasmid pcDNA3.1(+)-bot1+ in a TNT T7 Quick Coupled Transcription/Translation System (Promega) and added to the Tea1p-bound Ni²⁺-NTA magnetic agarose beads. The beads were washed in phosphate buffer and then eluted by boiling in SDS loading buffer for SDS-PAGE analysis.

To test for in vitro interaction between Bot1p and Orb6p, a GST-tagged ³⁵S_labeled Orb6p was expressed from plasmid pcDNA3.1(+)-GST-orb6+ in the TNT T7 System. The GST-Orb6p was bound to glutathione resin (Molecular Probes) and washed with a phosphate buffer (80 mM Na₂HPO₄, 20 mM NaH₂PO₄, 100 mM NaCl, pH 7.5). ³⁵S-labeled Bot1p was then added to the GST-Orb6p-bound glutathione resin and then washed with the phosphate buffer. The resin was then eluted by boiling in SDS loading buffer and proteins were separated by SDS-PAGE.

Assay of Orb6p Protein Kinase Activity

GST-tagged Orb6p was purified by glutathione resin (Molecular Probes) from the strains FV17p or FV194p co-expressing GST-Orb6p in the presence or absence of HA-Bot1p, respectively. The GST-Orb6 bound resin was then washed with kinase assay buffer (50 mM Tris-HCl, pH 7.5, 100 mM NaCl, 10 mM MgCl₂, 1 mM MnCl₂), and resuspended in 25 μl of kinase buffer containing 10 μCi of [γ-³²P] ATP (6000 Ci/mmol), and 20 μM ATP. The kinase assay was terminated after 30 min at 30° C. and the products were separated by gel electrophoresis. Orb6p phosphorylation was quantified using ImageQuant (Molecular Dynamics), and calibrated for comparison based on protein amounts determined by Western blot analysis using NIH Image.

Orb6p Protein Kinase is Associated with Novel Protein Bot1p

To identify novel components of the Orb6p pathway, a two-hybrid screen was performed to identify cDNAs encoding proteins that interact with Orb6p. One of these, isolated 22 times in this screen, was a novel protein that we named Bot1p. Histidine prototrophy (i.e., growth assay in the absence of histidine) demonstrated the activation of the HIS3-GAL4 promoter. Beta-galactosidase activity showed the LACZ-GAL4 promoter was being transcribed. The specificity of the positive interaction between Orb6p and Bot1p in the two-hybrid system was controlled by cotransformation of pAS1-orb6 and pACT-bot1 independently with a number of control plasmids. Co-expression of SNF1p or SNF4p was used as positive controls. Orb6p was unable to support the activation of the LACZ-GAL4 and HIS3-GAL4 promoters when co-expressed with GAL4 activation domain fused to SNF4p, or with the GAL4 activation domain (GAD) alone. Bot1p was unable to support the activation of the LACZ-GAL4 and HIS3-GAL4 promoters when co-expressed with the GAL4 binding domain fused to p53, Cdc23p, GAL4 DNA binding domain (GBD) alone, or fused to SNF1p or Cdt1p.

To confirm this interaction between Bot1p and Orb6p in fission yeast extracts, HA-Bot1p and GST-Orb6p were co-expressed in fission yeast cells, and GST-Orb6p was purified using glutathione-coupled resin. HA-Bot1p co-purified with GST-Orb6p, but did not co-purify with the resin when co-expressed with GST alone. Furthermore, neither GST nor GST-Orb6p co-purified with HA-tagged Cdc23p, when co-expressed as an additional control for specificity. These results indicate that Bot1p bind to Orb6p in fission yeast extracts.

Bot1p Performs an Essential function and Has a Role in the Control of Cell Shape

The bot1 gene (GenBank accession number AF352796) encodes a novel 315 amino acid protein. A BLAST search (NCBI) using default parameters identified two homologs: in Saccharomyces cerevisiae (YGR165w; 23% identity and 43% similarity over 270 amino acids of alignment) and in Candida albicans (SDSTC5476/Contig5-3220; 24% identity, 43% similarity over 201 amino acids). The C. albicans and S. cerevisiae homologs are closer in similarity to each other (39% identity and 54% similarity over the whole length) then to S. pombe Bot1p. A homolog has not been identified in Aspergillus. An alignment of the Bot1 protein (Bot1 p) from these fungi is shown in the FIGURE. The S. pombe and C. albicans Bot1 genes do not have introns, but the S. cerevisiae Bot1 gene contains one intron.

To investigate the function of Bot1p in cell morphogenesis of fission yeast, the entire open reading frame of bot1 was deleted. Tetrads were dissected on yeast extract plates and spores were germinated at 32° C. Following germination, spores deleted for the bot1+ gene (bot1::ura4+ ade6-leu1-32 ura4-D18) produced inviable microcolonies with characteristically bottle-shaped cells. This phenotype and the fact that Bot1p is a binding component of Tea1p (see below) suggested the name bot1. bot1Δ cells appeared smaller and rounder 24 hr after germination, while wild-type 972 cells formed a small colony of healthy cells. bot1Δ cells stopped dividing and displayed a round or bottle-shaped form 48 hr after germination, while the wild-type cells formed a normal size colony. These findings indicate that Bot1p is essential and is required for maintenance of normal cell shape.

To analyze the effects of loss of Bot1p, one copy of bot1+ under the control of the thiamine-repressible nmt1 promoter was integrated in the bot1Δ strain (FV2p). Addition of thiamine to the growth medium represses the activity of the full-strength nmt1 promoter, although it does not completely shut it off (16, 47). Mutant bot1Δ cells containing integrated bot1+ under control of the nmt1 promoter were grown exponentially for at least eight generation at 32° C., then thiamine was added to the culture. Cell density was never allowed to exceed 0.5 O.D. Cells were fixed at the appropriate times and stained with calcofluor to visualize the cell wall, DAPI to visualize the cell nucleus, and anti-tubulin, anti-actin or anti-Orb6p antibody.

When the promoter was active in medium lacking thiamine, bot1Δ cells were viable and were similar in shape and growth rate to wild-type 972 cells. The shape of bot1Δ cells began to change 26 hr after thiamine addition at 32° C., as cells became shorter. After 44 hr of culture in medium containing thiamine, bot1Δ cells were rounded or bottle shaped and tended to grow with only one tip. This was deduced to be the old end by observing pairs of daughter cells. These cells displayed a very similar phenotype to the one observed following germination of the bot1Δ strain. Forty-four hours after thiamine addition, 78% of the cells grew with one tip and 13% with two tips, while 4% were still dividing. Five percent of the cells were completely round at this point.

Effects of decreased levels of Bot1p on the microtubule and actin cytoskeletons were analyzed under different conditions: wild-type 972 cells, bot1A leu2+::nmt1-bot1+ cells grown in the absence of thiamine, and bot1Δ leu2+::nmt1-bot1+ cells grown for 44 hr after thiamine addition. A decrease in Bot1p levels profoundly affected the distribution of the actin cytoskeleton, and that actin was only localized at one tip, where actin patches also appear to be less defined. In contrast, the distribution of microtubules did not appear altered by the decrease of Bot1p levels.

Altered Bot1p levels were also found to have an effect on the localization of Orb6p. In wild-type 972 cells, Orb6p localized at the cell septum, and at one or both tips in cells growing from one end or two, respectively (62). In bot1Δ leu2+::nmt1-bot1+ cells grown in the absence of thiamine, Orb6p was found predominantly at one end, although in most cells it was observed in reduced amounts at the second tip. In bot1Δ cells grown in the presence of thiamine, Orb6p completely disappeared from the new end and was only found at the single growing tip (the old end). These results indicate that decreased levels of Bot1p alter the localization of Orb6p and actin.

Bot1p Localizes to the Cell Tips Similarly to Orb6p

To study the intracellular localization of Bot1p, cells expressing doubly Myc-tagged Bot1p were stained with an anti-Myc antibody. Myc₂-bot1+ was moderately expressed from a Rep41 plasmid under the control of an attenuated nmt1 promoter (10). The Myc₂-bot1+ construct was able to rescue the lethality of the bot1 deletion strain, indicating that the tag is not altering the functionality of the Bot1p. Myc-Bot1p was found localized in cytoplasmic dots, which aggregated at the cell tips during interphase. Myc-Bot1p was found at one tip in cell growing in a monopolar fashion and at both tips in cells growing from both ends, as determined by calcofluor staining. When a strain was produced where the endogenous bot1 gene was fused to a sequence encoding GFP, the fluorescent signal was found to be extremely faint although, when observable, its localization close to the tips was consistent with the results presented above.

Bot1p, Orb6p and actin were also found localized to the growing cell tips. The endogenous orb6 gene was fused to a sequence encoding a triple HA tag. Both Bot1p and Orb6p localized to the same cell tips. A similar pattern of localization was also observed when Myc-Bot1p and actin were compared. Thus, our results indicate that the timing and pattern of Bot1p localization are very similar to Orb6p and actin.

Bot1 and Orb6 Shows Genetic Interaction with Tea1

To understand the function of Bot1p in the control of cell polarity, genetic interactions with various genes important for cell morphology were assessed. Firstly, Bot1p overexpression was assessed to determine if it could suppress any of the known orb, tea or ban mutants, which define 19 independent genes involved in different aspects of cell morphogenesis (61). Bot1p overexpression could only suppress the morphological phenotype of orb11, a gene that when mutated leads to loss of cell polarity, cell wall weakening, and ultimately cell lysis (61). Interestingly, orb11 is also the only mutant gene that, to our knowledge, can be suppressed by Orb6p overexpression. This suppression is specific to Bot1p and Orb6p overexpression since expression in orb11 of other known morphological genes (e.g., ras1+, ral1/scd1+, ral3/scd2+, kin1+, pck1+, pck2+, cdc42+, ppe1+ and pypl+, see ref. 56) did not alleviate any of its defects (61). This observation supports the concept that orb6 and bot1 share a common function in the control of cell morphology.

Secondly, bot1Δ mutants were assessed for synthetic interactions with any of the known orb, tea or ban mutants. The bot1Δ mutants, which normally retain shape at one tip, completely lost growth polarity when the tea1 gene was mutated. Tea1p functions as a positional marker for cell growth and is delivered to the cell tips by microtubules (40). Tea1p is not essential, since tea1-1 and tea1Δ cells are viable at all temperatures, although they show a defect in the spatial control of polarized cell growth. At 25° C., the mutant cells display a normal shape although the usual order of tip activation is altered and they fail to activate growth from the second tip (18, 61). At 36° C., tea1-1 and tea1Δ cells bend and branch because cell growth is started at improper locations (40, 61).

A double mutant tea1-1 bot1Δ carrying an integrated copy of bot1+ under the control of the thiamine-repressible nmt1 promoter was constructed. Cells were grown in the presence of thiamine at 25° C. for 25 hr to switch off the nmt1 promoter, then exponentially grown at 36° C. for another 12 hr. Double mutant cells were compared to wild-type 972 cells, bot1Δ leu2+::nmt1-bot1+ cells, and tea1-1 cells grown in the same fashion. About 83% of cells of the double mutant tea1-1 bot1Δ leu2+::nmt1-bot1+ appeared completely round. Conversely, cells of the single mutant bot1Δ leu2+::nmt1-bot1+ still mostly grew in a polarized fashion from one tip (72%). Mutant tea1-1 cells showed cell bending and branching in 82% of cells but always grew in a polarized fashion. These findings indicate that Tea1p contributes to maintenance of cell polarity when the levels of Bot1p are limiting.

Interestingly, tea1-1 mutants are synthetically lethal with mutations in the orb6 gene. Mutant tea1-1 cells were viable at 36° C. like the wild-type 972 cells. Similarly, orb6Δ mutants expressing low levels of Orb6p were viable in the presence of thiamine at 36° C., although the mutant cells were rounded (62). Conversely, tea1-1 orb6Δ cells grew very poorly when Orb6p levels decreased indicating that tea1 mutations are synthetically lethal with reduced levels of Orb6p.

Consistent with the concept that Tea1p may affect in Orb6p and Bot1p functions, Bot1p and Orb6p localization is altered in tea1Δ and tea1-1 mutants. As discussed above, Bot1p and Orb6p were present at one tip during monopolar growth, and at both tips during bipolar growth. In tea1Δ mutants at 36° C., however, Bot1p and Orb6p were found in abnormal positions within the cell in 33% of cells. In the remaining cells at both 25° C. and 36° C., Bot1p and Orb6p were always found at one single growing tip, indicating that Tea1p function is also required at all temperatures for their localization to the second cell end. These results show that the normal localization of Bot1p and Orb6p to the correct position at the cell tips is altered in tea1 mutants, indicating that Bot1p and Orb6p localization may be dependent on Tea1p function.

To determine the effect of Bot1p on Tea1p, the latter protein was localized by a polyclonal anti-Tea1p antibody in bot1Δ leu1+::nmt1-bot1+ cells grown in the presence of thiamine for 44 hr, and thus expressing Bot1p at reduced levels. Altering Bot1p levels did not affect Tea1p localization, which is maintained on both cell tips in bot1Δ cells grown in the absence or presence of thiamine. These results indicate that Bot1p does not affect Tea1p localization, but has a role in the control of Orb6p. Since Bot1p localization is altered in tea1Δ mutants, these findings also indicate that Tea1p, Bot1p and Orb6p may be functioning in hierarchical order, with Tea1p determining the location of Bot1p and Bot1p, in turn, the location of Orb6p.

Two-Hybrid Screen Identifies Bot1p as a Protein Interacting with Tea1p

To understand the mechanism of Tea1p-dependent control of cell polarity, a two-hybrid screen was performed to identify cDNAs encoding proteins that interact with Tea1p. One of the proteins identified in the two-hybrid screen for Tea1p-associated components was Bot1p. Bot1p was isolated eight times in the screen. The specificity of the interaction between Bot1p and Tea1p was tested in control experiments. Tea1p, fused to the GAL4 binding domain, was able to activate the HIS3-GAL4 and the LACZ-GAL4 promoters when co-expressed with Bot1p fused to the GAL4 activation domain. Co-expressing pAS1-SNF1 and pACT-SNF4 was the positive control. Tea1p was unable to support the expression of HIS3 and LACZ when co-expressed with the GAL4 activation domain alone (GAD), or with GAL4 activation domain fused to SNF4. Bot1p was unable to support the activation of the HIS3-GAL4 and LACZ-GAL4 promoters when co-expressed with the GAL4 binding domain fused to Cdc23p, p53, SNF1 or Cdt1p, or with the GAL4 DNA binding domain alone (GDB).

To confirm this interaction between Tea1p and Bot1p in fission yeast extracts, GST-Tea1p and HA-Bot1p were co-expressed in fission yeast cells, and GST-Tea1p was purified using glutathione-coupled resin. The beads were further incubated with an extract containing HA-Bot1p or HA-Cdc23p as a control. Consistent with Tea1p associating with Bot1p, we found that HA-Bot1p co-purified with GST-Tea1p, but did not with GST alone. HA-Cdc23p did not co-purify with either GST or GST-Tea1p. These results indicate that Bot1p associates with Tea1p in fission yeast cell extracts.

Role of Microtubules in Bot1p Localization

Because our previous results indicated a role for Tea1p in the control of Bot1p function, the effect of microtubule depolymerization on Bot1p localization was assessed. Tea1p is dependent on the microtubule cytoskeleton for delivery to the cell tips, and microtubule depolymerization leads to Tea1p delocalization (40). Cells expressing Myc-Bot1 were exposed to the microtubule-depolymerizing drug MBC (methyl-benzidazole-carbamate) for 45 min. This treatment led to virtually complete microtubule depolymerization. The control, treated for the length of same time with the same amount of DMSO, displayed an intact microtubule cytoskeleton and normal Bot1p localization at the cell tips. When microtubules are depolymerized, Bot1p-containing spots appeared more spread out and less restricted to the cell tips, although most of Bot1p was still found close to the cell surface. These observations indicate that the microtubule cytoskeleton has a role in confining the localization of Bot1p to the cell tips.

Role of Bot1p in the Interaction with Tea1p and Orb6p

To investigate whether Bot1p is able to form a complex with Tea1p and Orb6p, we determined if these proteins co-immunoprecipitate in cell extracts, if they interact in the yeast two-hybrid system, and if they are able to associate in vitro. Thus, HA-Bot1p was co-expressed with GST or with GST-Orb6p in fission yeast cells, and purified GST-Orb6p or GST using a glutathione-coupled resin. After binding GST or GST-Orb6p to glutathione beads, the beads were further incubated with an extract containing HA-Tea1p or HA-Cdc23p as a control. As shown before, HA-Bot1p co-purified with GST-Orb6p but not with GST. GST-Orb6p and HA-Bot1p did not co-purify with HA-Tea1p. HA-Cdc23p did not co-purify with either GST or GST-Orb6p.

Activation of the LACZ-GAL4 promoter was assayed to detect positive interaction between Orb6p and Tea1p in the presence or absence of Bot1p (i.e., yeast three-hybrid system). pACT2-tea1 and pAS1-orb6 were transformed into BY3161 along with the bot1 gene on a yeast plasmid carrying the Ura3+ marker (pDela bot1+). A Ura3+ plasmid (pDela) without insert was used as a control. Other controls were the positive interaction between Snf1p and Snf4p and the negative interaction between Orb6p and Snf4p. These results suggest that Bot1p does not associate with Tea1p and Orb6p in a ternary complex under these conditions, and that the interaction of Bot1p to Orb6p or Tea1p may be mutually exclusive.

To determine if the interaction of Bot1p with Tea1p or Orb6p is direct, the amino terminus of Tea1p (i.e., amino acid residues 1 to 490) fused to a His-tag (N-Tea1p) was bacterially expressed. The N terminus of Tea1p, containing the kelch repeats but not the coil-coiled domain, is sufficient for the interaction of Bot1p with Tea1p in the two-hybrid system. N-Tea1p was affinity purified using Ni²⁺-NTA resin, and the resin was then further incubated with ³⁵S-labeled Bot1p expressed in vitro using reticulocyte lysate. Bot1p can bind directly to the N terminus of Tea1p. To test the interaction between Bot1p and Orb6p, ³⁵S-labeled GST and GST-Orb6p were expressed in vitro using reticulocyte lysate and purified using glutathione-coupled resin. The resin was then further incubated with ³⁵S-labeled Bot1p which was also expressed in vitro using reticulocyte lysate. Bot1p did not co-purify with in vitro expressed Orb6p. These observations indicate that while the interaction of Tea1p with Bot1p is direct, the association of Bot1p with Orb6p may require at least another factor or protein modification (see below).

Finally, it was determined whether altering Bot1p levels has an effect on Orb6p protein kinase activity, as measured by autophosphorylation. GST-Orb6p was purified from a control strain or a strain overexpressing Bot1p, and in vitro protein kinase activity was assayed. Autoradiography was used to visualize ³²P-labeling of purified GST-Orb6p; GST-Orb6p was visualized by Western blotting with an anti-GST antibody. The average phosphorylation levels were measured in three independent trials. The level of GST-Orb6p autophosphorylation was the same for proteins isolated from control and Bot1p-overexpressing cells, indicating that an increase in Bot1p levels does not modulate Orb6p protein kinase activity.

Orb6p-associated Bot1p was not phosphorylated in vitro by Orb6p, indicating that Bot1p does not function as an Orb6p kinase substrate. Bot1p overexpression could not suppress the phenotype of orb6A or orb6-25 mutants, indicating that Bot1 is unlikely to function as an Orb6p effector, and consistent with its function in Orb6p localization.

The microtubule cytoskeleton is fundamental to the establishment of spatial order in the cell, and controls a number of specialized cell functions, including cell locomotion and polarized cell growth. In Schizosaccharomyces pombe cells, the intimate connection between microtubules and polarized cell growth has been emphasized by the identification of Tea1p. Tea1p is a microtubule-binding protein that functions as a marker for cell polarity (61, 40), localizes to both cell tips in an actin-independent fashion, and is part of a large protein complex (40). The key role of Tea1p in the spatial control of cell polarity indicates that it may be responsible for the delivery and/or retention of polarity determinants at the cell tip. So far, the only protein thought to be part of the Tea1p complex is Bud6p, an actin-binding protein, although it is unclear if the interaction with Tea1p is direct (18).

We have identified a novel protein Bot1p which interacts physically and functionally with both Tea1p and Orb6p. Orb6p is a conserved ser/thr kinase that has a role in both cell polarity and cell cycle control (62). Bot1p is essential for cell viability, and is important for maintenance of cell shape and polarized cell growth. Bot1p associates with Tea1p in fission yeast extracts, in the two-hybrid system, and in vitro, and is dependent on Tea1p for its proper localization to the cell tips and to the new end. Furthermore, mutations in the tea1 gene substantially worsen the phenotype of bot1Δ mutants when Bot1p expression levels are reduced. Conversely, Tea1p remains associated at both cell tips in cells expressing decreased levels of Bot1p.

Our results strongly suggest a functional interaction between Orb6p and Bot1p. First, we found that Orb6p and Bot1p associate in the two-hybrid system as well as in cell extracts. Second, we observed that a decrease in the intracellular levels of Bot1p disrupt the normal localization of Orb6p. Third, Orb6p and Bot1p show similar localization patterns in fission yeast cells. Finally, we found that Bot1p and Orb6p over-expression specifically suppresses the phenotype of mutants in one of the orb genes, orb11-59, while it does not have any obvious effects on the mutant phenotype of other orb genes. orb11 is one of the 19 genes that were identified in a screen for morphological mutants, together with orb6 and tea1 (62).

Thus, these observations indicate that Bot1p functions as a molecular connection between the early establishment of cell polarity mediated by Tea1p, and components of the signaling pathways involved in cytoskeleton reorganization and cell growth. Tea1p is dependent on microtubules for its continued delivery to the cell tips, and, consistent with its proposed role in the organization of Bot1p, microtubule depolymerization subtly affects Bot1p, leading to a more widespread localization at the cell periphery. It is important to note, however, that Tea1p and microtubules are not essential for cell growth. tea1Δ cells grow in a polarized fashion and are viable at all temperatures, although they tend to become rather branched and deformed at 36° C. (40). Microtubule depolymerization does not substantially hinder tip extension during interphase (see 25). In the absence of Tea1p, many conserved components essential for polarized cell growth, such as actin, Rho1p, Ssp1p, Bud6p, Orb6p, as well as Bot1p, are still delivered to at least one of the cell tips (2; 18, 52, results herein). Instead, Tea1p plays an important part in the spatial organization of such molecules. Specifically, Tea1p has a role in defining the cell ends and in restricting cell growth to the cell tips. This function is particularly important at higher temperatures, and may reflect a role of Tea1p in the organization and stability of large multimolecular complexes at the cell tips. Furthermore, Tea1p is required for bipolar growth at all temperatures, indicating that it may also have a regulatory role in the activation of the second tip.

How could Tea1p fulfill the role of a macromolecular organizer? Tea1p is part of a family of proteins that share a kelch repeats-containing “propeller” domain (1). Proteins containing such motifs are thought to have a general role in the organization of multimolecular complexes and to have diverse functions, including the association with the actin cytoskeleton, and the control of cell morphology (1). The specific structure of the propeller domain in Tea1p may allow multiple protein-protein interactions, indicating that different effectors may even bind the same Tea1p molecule (1). Consistent with the potential complexity of its function, Tea1p is part of a large 1000 kDa complex (40). It is interesting and consistent with the proposed role of Bot1p as one of the Tea1p effectors that the Tea1p propeller domain directly interacts in vitro with Bot1p. Interestingly, Tea1p is found at both tips even during monopolar growth, while Bot1p localizes to the second tip only when cell growth is activated. This finding indicates that the Tea1p-Bot1p interaction is regulated during the cell cycle.

How does Bot1p control Orb6p localization? Orb6p, like Bot1p, is essential for cell growth and, unlike tea1Δ mutants, orb6Δ and bot1Δ mutants are inviable at all temperatures. Importantly, although microtubules and Tea1p are required to set up correctly the growth zone along the cell axis, they are not needed once polarized cell growth is established. These observations indicate that components of the growth zone can associate and remain functional in the absence of Tea1p and microtubules. Indeed, Orb6p and Bot1p still co-localize in tea1Δ mutants, even in abnormal locations, indicating that Bot1p and Orb6p can associate also in the absence of Tea1p. Furthermore, Bot1p does not form a trimeric complex with Tea1p and Orb6p, indicating that it may bind Orb6p in a second step. Thus, it is possible that Tea1p is involved in the organization of an initial complex of proteins at the cell tips and that this structure then functions as an adaptor, binding other components that regulate activation of cell growth. Bot1p may be involved in the localization of other proteins, in addition to Orb6p. Indeed, the lethality of bot1Δ strains is not suppressed by Orb6p overexpression, indicating that Bot1p may have additional functions or that it is required for Orb6p activity.

Interestingly, in vitro expressed Orb6p and Bot1p do not bind, showing that the association of additional proteins or specific protein modifications may be necessary. Orb6p is phosphorylated in vivo and associates with another protein Mob2p. Mob2 is related to S. cerevisiae Mob1p, which binds to Dbf2p kinase and allows its activation by the upstream kinase Cdcl5p, by phosphorylation of two sites conserved in the Orb6p kinase family (36). Bot1p association to Orb6p may be dependent on Mob2p-dependent phosphorylation of Orb6p kinase.

In summary, our findings identify a novel protein Bot1p which closely associates with Tea1p and functions as a Tea1p effector in the control of polarized cell growth, providing an insight into the molecular mechanism of Tea1p- and microtubule-dependent cell polarity control. Furthermore, a specific mechanism allowing the localization of Orb6p kinase to the cell tips is described.

Table of S. pombe Strains

| Strain | Genotype | Source |
|---|---|---|
| FV16p | ade6-704 leu1-32 ura4-D18 h- pESP-GST pRep41-HA₃bot1 | Verde et al. |
| FV17p | ade6-704 leu1-32 ura4-D18 h- pESP-GSTOrb6+ pRep41-HA₃bot1 | Verde et al. |
| FV194p | ade6-704 leu1-32 ura4-D18 h- pESP-GSTOrb6+ pRep41 | Verde et al. |
| FV366p | ade6-704 leu1-32 ura4-D18 h- pESP-GST pRep41-HA₃cdc23 | Verde et al. |
| FV367p | ade6-704 leu1-32 ura4-D18 h- pESP-GSTOrb6+ pRep41-HA₃cdc23 | Verde et al. |
| FV567 | ade6-704 leu1-32 ura4-D18 h- | P. Nurse |
| FV1p | bot1+/bot1::ura4+ade6-M210/M216 leu1-32/leu1-32 ura4-D18/ura4-D18 h+/h- | Verde et al. |
| FV2p | bot1::ura4+ade-leu1-32 ura4-D18 - leu1+::nmt1-bot1+ | Verde et al. |
| FV362p | orb6-HA₃-sup3-5 ade6-704 leu1-32 ura4-D18 h- pRep41-myc₂bot1 | Verde et al. |
| FV364p | ade6-704 leu1-32 ura4-D18 h- pRep41- myc₂bot1 | Verde et al. |
| FV135p | orb11-59 ade6-M210 leu1-32 h- pRep3x | Verde et al. |
| FV136p | orb11-59 ade6-M210 leu1-32 h- Rep3x-bot1+ | Verde et al. |
| FV265p | orb11-59 ade6-M210 leu1-32 h- Rep3x-orb6+ | Verde et al. |
| PN1 | 972 h- | P. Nurse |
| FV1 | tea1-1 ade6-M210 leu1-32 h- | Verde et al. |
| FV120p | tea1-1 bot1::ura4+ ade- leu1-32 ura4-D18 leu1+:: nmt1-bot1+ | Verde et al. |
| FV338 | orb6::ura4+ ade- leu1-32 ura4D18 h⁹⁰ leu1+:: nmt1-orb6+ | (53) |
| FV487 | tea1-1 orb6::ura4+ ade- leu1-32 ura4D-18 leu1+:: nmt1-orb6+ | Verde et al. |
| FV368 | tea1Δ::ura4+ orb6-HA₃-sup3-5 ade6-M210 ura4D-18 h+ pRep41-myc₂bot1 | Verde et al. |
| FV308p | ade6-704 leu1-32 ura4-D18 h- pESP-GST | Verde et al. |
| FV307p | ade6-704 leu1-32 ura4-D18 h- pESP-GSTtea1+ | Verde et al. |
| FV372p | ade6-704 leu1-32 ura4-D18 h- pRep41-HA₃cdc23 | Verde et al. |
| FV370p | ade6-704 leu1-32 ura4-D18 h- pRep41-HA₃bot1 | Verde et al. |
| FV371p | ade6-704 leu1-32 ura4-D18 h- leu1+::nmt1-HA₃tea1 | Verde et al. |

### REFERENCES

1. Adams J, Kelso R, Cooley L (2000) The kelch repeat superfamily of proteins: Propellers of cell function. Trends Cell Biol. 10,17-24.

2. Arellano M, Duran A, Perez P (1997) Localisation of the Schizosaccharomyces pombe rho1p GTPase and its involvement in the organisation of the actin cytoskeleton. J. Cell Sci. 110, 2547-2555.

3. Aves S J, Tongue N, Foster A J, Hart E A (1998) The essential Schizosaccharomyces pombe cdc23 DNA replication gene shares structural and functional homology with the Saccharomyces cerevisiae DNA43 (MCM10) gene. Curr. Genet. 34, 164-171.

4. Bahler J, Pringle JR (1998) Pom1, a fission yeast protein kinase that provides positional information for both polarized cell growth and cytokinesis. Genes Dev. 12, 1356-1370.

5. Bidlingmaier S, Weiss E L, Seidel C, Drubin D G, Snyder M (2001) The Cbk1p pathway is important for polarized cell growth and cell separation in Saccharomyces cerevisiae. Mol Cell Biol. 7, 2449-2462.

6. Brook J D, McCurrach M E, Harley H G, Buckler A J, Church D, Aburatani H, Hunter K, Stanton V P, Thirion J P, Hudson T, et al. (1992) Molecular basis of myotonic dystrophy: Expansion of a trinucleotide (CTG) repeat at the 3′ end of a transcriptencoding a protein kinase family member. Cell 68, 799-808 and 69, 385.

7. Browning H, Hayles J, Mata J, Aveline L, Nurse P, McIntosh J R (2000) Tea2p is a kinesin-like protein required to generate polarized growth in fission yeast. J. Cell Biol. 151, 15-28.

8. Brunner D, Nurse P (2000) CLIP170-like tip1p spatially organizes microtubular dynamics in fission yeast. Cell 102, 695-704.

9. Chang E C, Barr M, Wang Y, Jung V, Xu H P, Wigler M H (1994) Cooperative interaction of S. pombe proteins required for mating and morphogenesis. Cell 79, 131-141.

10. Craven R A, Griffiths D J, Sheldrick K S, Randall R E, Hagan I M, Carr A M (1998) Vectors for the expression of tagged proteins in Schizosaccharomyces pombe. Gene 221, 59-68.

11. Cvrckova F, De Virgilio C, Manser E, Pringle J R, Nasmyth K (1995) Ste20-like protein kinases are required for normal localization of cell growth and for cytokinesis in budding yeast. Genes Dev. 9, 1817-1830.

12. Drummond D R, Cross R A (2000) Dynamics of interphase microtubules in Schizosaccharomyces pombe. Curr. Biol. 10, 766-775.

13. Durrenberger F, Kronstad J (1999) The ukc1 gene encodes a protein kinase involved in morphogenesis, pathogenicity and pigment formation in Ustilago maydis. Mol. Gen. Genet. 2, 281-289.

14. Durfee T, Becherer K, Chen P L, Yeh S H, Yang Y, Kilburn A E, Lee W H, Elledge S J (1993) The retinoblastoma protein associates with the protein phosphatase type 1 catalytic subunit. Genes Dev. 7, 555-569.

15. Fantes P, Nurse P (1977) Control of cell size at division in fission yeast by a growth-modulated size control over nuclear division. Exp. Cell Res. 107, 377-386.

16. Forsburg S L (1993) Comparison of Schizosaccharomyces pombe expression systems. Nucl. Acids Res. 21, 2955-2956.

17. Gilbreth M, Yang P, Wang D, Frost J, Polyerino A, Cobb M H, Marcus S (1996) The highly conserved skb1 gene encodes a protein that interacts with Shkl, a fission yeast Ste20/PAK homolog. Proc. Natl. Acad. Sci. USA 93, 13802-13807.

18. Glynn J M, Lustig R J, Berlin A, Chang F (2001) Role of bud6p and tea1p in the interaction between actin and microtubules for the establishment of cell polarity in fission yeast. Curr. Biol. 11, 836-845.

19. Goode B L, Drubin D G, Barnes G (2000) Functional cooperation between the microtubule and actin cytoskeletons. Curr. Opin. Cell Biol. 12, 63-71.

20. Gundersen G G, Cook T A (1999) Microtubules and signal transduction. Curr. Opin. Cell Biol. 11, 81-94.

21. Hagan I M, Hyams J S (1988) The use of cell division cycle mutants to investigate the control of microtubule distribution in the fission yeast Schizosaccharomyces pombe. J. Cell Sci. 89, 343-357.

22. Hagan I (1998) The fission yeast microtubule cytoskeleton. J. Cell Sci. 111, 1603-1612.

23. Hall A (1998) G proteins and small GTPases: distant relatives keep in touch. Science 280, 2074-2075. 24. Harold F M (1990) To shape a cell: an inquiry into the causes of morphogenesis of microorganisms. Microbiol. Rev. 54, 381-431.

25. Hayles J, Nurse P (2001) A journey into space. Nat. Rev. Mol. Cell Biol. 2, 647-656.

26. Hiraoka Y, Toda T, Yanagida M (1984) The NDA3 gene of fission yeast encodes beta-tubulin: a cold-sensitive nda3 mutation reversibly blocks spindle formation and chromosome movement in mitosis. Cell 39, 349-358.

27. Hirata D, Nakano K, Fukui M, Takenaka H, Miyakawa T, Mabuchi I (1998) Genes that cause aberrant cell morphology by overexpression in fission yeast: a role of a small GTP-binding protein Rho2 in cell morphogenesis. J. Cell Sci. 111, 149-159.

28. Hofmann J F, Beach D (1994) cdt1 is an essential target of the Cdc10/Sct1 transcription factor: requirement for DNA replication and inhibition of mitosis. EMBO J. 13, 425-434.

29. Ishii K, Kumada K, Toda T, Yanagida M (1996) Requirement for PP1 phosphatase and 20S cyclosome/APC for the onset of anaphase is lessened by the dosage increase of a novel gene sds23+. EMBO J. 15, 6629-6640.

30. Ishizaki T, Maekawa M, Fujisawa K, Okawa K, Iwamatsu A, Fujita A, Watanabe N, Saito Y, Kakizuka A, Morii N, Narumiya S (1996) The small GTP-binding protein Rho binds to and activates a 160 kDa Ser/Thr protein kinase homologous to myotonic dystrophy kinase. EMBO J. 15, 1885-1893.

31. Ishizaki T, N. M., Fujisawa K, Maekawa M, Watanabe N, Saito Y, Narumiya S (1997) p160ROCK, a Rho-associated coiled-coil forming protein kinase, works downstream of Rho and induces focal adhesions. FEBS Lett. 404, 118-124.

32. Justice R W, Zilian 0, Woods D F, Noll M, Bryant P J (1995) The Drosophila tumour suppressor gene warts encodes a homolog of human myotonic dystrophy kinase and is required for the control of cell shape and proliferation. Genes Dev. 9, 534-546.

33. Leung T, Manser E, Tan L, Lim L (1995) A novel serine/threonine kinase binding the Ras-related RhoA GTPase which translocates the kinase to peripheral membranes. J. Biol. Chem. 270, 29051-29054.

34. Leung T, Chen X Q, Manser E, Lim L (1996) The p160 RhoA-binding kinase ROK alpha is a member of a kinase family and is involved in the reorganization of the cytoskeleton. Mol. Cell. Biol. 16, 5313-5327

35. Mackay D J, Esch F, Furthmayr H, Hall A (1997) Rho- and Rac-dependent assembly of focal adhesion complexes and actin filaments in permeabilized fibroblasts: an essential role for ezrin/radixin/moesin proteins. J. Cell Biol. 138, 927-938.

36. Mah A S, Jang J, Deshaies R J (2001) Protein kinase Cdcl5 activates the Dbf2-Mob1 kinase complex. Proc. Natl. Acad. Sci. USA 98, 7325-7330.

37. Marcus S, Polyerino A, Chang E, Robbins D, Cobbs M H, Wigler M (1995) Shk1, a homolog of the Saccharomyces cerevisiae Ste20 and mammalian p65 pak protein kinases, is a component of the Ras/Cdc42 signaling module in the fission yeast Schizosaccharomyces pombe. Proc. Natl. Acad. Sci. USA 92, 6180-6184.

38. Marks J, Hyams J S (1985) Localization of F-actin through the cell division cycle of Schizosaccharomyces pombe. Eur. J. Cell Biol. 110, 417-425.

39. Marks J, Hagan I M, Hyams J S (1986) Growth polarity and cytokinesis in fission yeast: the role of the cytoskeleton. J. Cell Sci. Suppl. 5, 229-241.

40. Mata J, Nurse P (1997) tea1 and the microtubular cytoskeleton are important for generating global spatial order within the fission yeast cell. Cell 89, 939-949.

41. Matsui T, Amano M, Yamamoto T, Chihara K, Nakafuku M, Ito M, Nakano T, Okawa K, Iwamatsu A, Kaibuchi K (1996) Rho-associated kinase, a novel serine/threonine kinase, as a putative target for small GTP binding protein Rho. EMBO J. 15, 2208-2216.

42. Matsui T, Maeda M, Doi Y, Yonemura S, Amano M, Kaibuchi K, Tsukita S, Tsukita S (1998) Rho-kinase phosphorylates COOH-terminal threonines of ezrin/radixin/moesin (ERM) proteins and regulates their head-to-tail association. J. Cell Biol. 140, 647-657.

43. Miller P J, Johnson D I (1994) Cdc42p GTPase is involved in controlling polarized cell growth in Schizosaccharomyces pombe. Mol. Cell. Biol. 14, 1075-1083.

44. Millward T, Cron P, Hemmings B A (1995) Molecular cloning and characterization of a conserved nuclear serine(threonine) protein kinase. Proc. Natl. Acad. Sci. USA 92, 5022-5026.

45. Mitchison J M, Nurse P (1985) Growth in cell length in the fission yeast Schizosaccharomyces pombe. J. Cell Sci. 75, 357-376.

46. Moreno S, Klar A, Nurse P (1991) Molecular genetic analysis of fission yeast Schizosaccharomyces pombe. Meth. Enzymol. 194, 795-823.

47. Moreno M B, Duran A, Ribas J C (2000) A family of multifunctional thiamine-repressible expression vectors for fission yeast. Yeast 16, 861-872.

48. Nurse P (1975) Genetic control of cell size at cell division in yeast. Nature 256, 547-551.

49. Ottilie S, Miller JP, Johnson D I, Creasy C L, Sells M A, Bagrodia S, Forsburg S L, Chernoff J (1995) Fission yeast pak1+ encodes a protein kinase that interacts with Cdc42p and is involved in the control of cell polarity and mating. EMBO J. 14, 5908-5919.

50. Pruyne D, Bretscher A (2000) Polarization of cell growth in yeast. J. Cell Sci. 113, 365-375 and 571-585

51. Racki W J, Becam A M, Nasr F, Herbert C J. (2000) Cbk1p, a protein similar to the human myotonic dystrophy kinase, is essential for normal morphogenesis in Saccharomyces cerevisiae. EMBO J. 19, 4524-4532.

52. Rupes I, Jia Z, Young P G (1993) Ssp1 promotes actin depolymerization and is involved in stress response and new end take-off control in fission yeast. Mol. Biol. Cell. 10, 1495-510.

53. Sawin K E, Nurse P (1998) Regulation of cell polarity by microtubules in fission yeast. J. Cell Biol. 142, 457-471.

54. Sawin K E (2000) Microtubule dynamics: The view from the tip. Curr. Biol. 10, R860-R862.

55. Streiblova E, Wolf A (1972) Cell wall growth during the cell cycle of Schizosaccharomyces pombe. Z. Allg. Mikrobiol. 12, 673-684.

56. Snell et al. (1993) Investigations into the control of cell form and polarity: The use of morphological mutants in fission yeast. Dev. Suppl. 289-299.

57. Snell V, Nurse P (1994) Genetic analysis of cell morphogenesis in fission yeast —A role for casein kinase 11 in the establishment of polarized growth. EMBO J. 13, 2066-2074.

58. Terada S, Hirokawa N (2000) Moving on to the cargo problem of microtubule-dependent motors in neurons. Curr. Opin. Neurobiol. 10, 566-573.

59. Umesono K, Toda T, Hayashi S, Yanagida M (1983) Cell division cycle genes nda2 and nda3 of the fission yeast Schizosaccharomyces pombe control microtubular organization and sensitivity to anti-mitotic benzimidazole compounds. J. Mol. Biol. 168, 271-284.

60. Van Aelst L, D'Souza-Schorey C (1997) Rho GTPases and signaling networks. Genes Dev. 11, 2295-2322.

61. Verde F, Mata J, Nurse P (1995) Fission yeast cell morphogenesis: Identification of new genes and analysis of their role during the cell cycle. J. Cell Biol. 131, 1-10.

62. Verde F, Wiley DJ, Nurse P (1998) Fission yeast orb6, a ser/thr protein kinase related to mammalian rho kinase and myotonic dystrophy kinase, is required for maintenance of cell polarity and coordinates cell morphogenesis with the cell cycle. Proc. Natl. Acad. Sci. USA 95, 7526-7531.

63. Verde, F (1998) On growth and form: Control of cell morphogenesis in fission yeast. Curr. Opin. Microbiol. 1, 712-718.

64. Verde F (2001) Cell polarity: A tale of two Ts. Curr. Biol. 11, R600-R602.

65. Vega L R, Solomon F (1997) Microtubule function in morphological differentiation: Growth zones and growth cones. Cell 89, 825-828.

66. Xu T, Wang W, Zhang S, Stewart R A, Yu W (1995) Identifying tumor suppressor in genetic mosaics: the Drosophila lats gene encodes a putative kinase. Development 121, 1053-1063.

67. Yaffe M P, Harata D, Verde F, Eddison M, Toda T, Nurse P (1996) Microtubules mediate mitochondrial distribution in fission yeast. Proc. Natl. Acad. Sci. USA 93, 11664-11668.

68. Yarden O, Plamann M, Ebbole D J, Yanofsky C (1992) cot-1, a gene required for hyphal elongation in Neurospora crassa, encodes a protein kinase. EMBO J. 11, 2159-2166.

69. Zallen J A, Peckol E L, Tobin D M, Bargmann C L (2000) Neuronal cell shape and neurite initiation are regulated by the ndr kinase SAX-1, a member of the Orb6/Cot-1/Warts serine/threonine kinase family. Mol. Biol. Cell 11, 3177-3190.

70. Zang J, Lautar S (1996) A yeast three-hybrid method to clone ternary protein complex components. Anal. Biochem. 242, 68-72.

Patents, patent applications, and other publications cited herein are incorporated by reference in their entirety.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. Any of the three transitions can be used to claim the invention.

It should be understood that an element described in this specification should not be construed as a limitation of the claimed invention unless it is explicitly recited in the claims. Thus, the claims are the basis for determining the scope of legal protection granted instead of a limitation from the specification which is read into the claims.

In contradistinction, the prior art is explicitly excluded from the invention to the extent of specific embodiments that would anticipate the claimed invention or destroy novelty. In certain embodiments, the genus of polynucleotides or polypeptides may be claimed with the proviso that native nucleic acids or proteins are excluded (e.g., having a nucleotide or amino acid sequence which is not given in the sequence listing). For example, the degeneracy of the genetic code may be used to provide a polynucleotide having a nucleotide sequence encoding SEQ ID NO:6, but which is not SEQ ID NO:5. Similarly, a fungal Bot1 polypeptide may be provided that is functionally equivalent but not identical to the C. albicans protein (e.g., at least 90% identical) by changing one or more of the amino acid residues of SEQ ID NO:6.

Moreover, no particular relationship between or among limitations of a claim is intended unless such relationship is explicitly recited in the claim (e.g., the arrangement of components in a product claim or order of steps in a method claim is not a limitation of the claim unless explicitly stated to be so). All possible combinations and permutations of the individual elements disclosed herein are considered to be aspects of the invention; similarly, generalizations of the invention's description are considered to be part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

What is claimed is:

1. An isolated fungal Bot1 polypeptide.

2. The fungal Bot1 polypeptide of claim 1, wherein it has an amino acid sequence selected from the group consisting of SEQ ID NO:2, SEQ ID NO:4, and SEQ ID NO:6.

3. A polypeptide fragment which has an amino acid sequence comprised of at least 10 contiguous amino acids from the fungal Bot1 polypeptide of claim 1.

4. An artificial substrate which has the polypeptide fragment of claim 3 stably immobilized thereon.

5. A fusion polypeptide which has an amino acid sequence comprised of (a) at least 10 contiguous amino acids from the fungal Bot1 polypeptide of claim 1 and (b) at least one heterologous amino acid sequence.

6. An isolated polypeptide complex comprised of the fungal Bot1 polypeptide of claim 1 and at least one of Tea1 polypeptide or Orb6 polypeptide.

7. An isolated polypeptide complex comprised of the fungal Bot1 polypeptide of claim 1, which is not comprised of Tea1 polypeptide and Orb6 polypeptide.

8. A specific binding molecule specific for at least the fungal Bot1 polypeptide of claim 1, which is not Orb6 polypeptide.

9. An isolated polynucleotide encoding the fungal Bot1 polypeptide of claim 1, or the complement thereof.

10. The polynucleotide of claim 9, wherein it has a nucleotide sequence selected from the group consisting of SEQ ID NO:1, SEQ ID NO:3, SEQ ID NO:5, and complementary sequences thereof.

11. A polynucleotide fragment which has a nucleotide sequence comprised of at least 15 contiguous nucleotides from the polynucleotide of claim 9.

12. An artificial substrate which has the polynucleotide fragment of claim 11 stably immobilized thereon.

13. A chimeric polynucleotide which has a nucleotide sequence comprised of (a) at least 15 contiguous nucleotides from the polynucleotide of claim 9 and (b) at least one heterologous nucleotide sequence.

14. The polynucleotide of claim 9, wherein it is at least partially double stranded.

15. An expression construct comprised of (a) at least the polynucleotide fragment of claim 11 and operably linked to (b) at least one transcriptional regulatory region.

16. The expression construct of claim 15, wherein it is at least partially double stranded.

17. A probe or primer specific for at least the polynucleotide of claim 9.

18. A cell comprised of a mutated Bot1 genetic locus or an ectopic Bot 1 gene, or extract produced therefrom.

19. A transfected cell or fungus comprised of the fusion polypeptide of claim 5, or extract produced therefrom.

20. A transfected cell or fungus comprised of the chimeric polynucleotide of claim 13, or extract produced therefrom.

21. A process for producing a fungal Bot1 polypeptide comprising:
(a) introducing an expression construct of claim 15 into a host cell or organism and
(b) isolating the fungal Bot1 polypeptide produced by the host cell or organism.

22. A process for specific binding a fungal Bot1 polypeptide comprising:
(a) providing a sample containing polypeptides and
(b) contacting the sample with a binding molecule specific for at least the fungal Bot1 polypeptide of claim 1 under conditions that result in specific binding with polypeptides in the sample.

23. A process of nucleic acid hybridization comprising:
(a) providing a sample containing polynucleotides and
(b) contacting the sample with a polynucleotide which has a nucleotide sequence comprised of at least 15 contiguous nucleotides from the polynucleotide of claim 9 under conditions that result in specific hybridization with polynucleotides in the sample.

24. A process of screening for an antifungal agent comprising:
(a) providing a library of candidate drugs,
(b) determining at least one activity of a fungal Bot1 polypeptide of claim 1 or variant thereof in the presence of a candidate drug,
(c) selecting at least one drug by its ability to at least affect one activity of the fungal Bot1 polypeptide or variant thereof, and
(d) confirming that the selected drug is an antifungal agent by its ability to at least slow growth or reduce viability of fungus.

25. The process of claim 24, wherein the selected drug at least increases or decreases specific binding between the fungal Bot1 polypeptide or variant thereof and Orb6 polypeptide.

26. The process of claim 24, wherein the selected drug at least decreases viability of a fungus.

27. The process of claim 24, wherein the selected drug at least decreases growth of a fungus.

28. The process of claim 24, wherein the selected drug at least alters control of cell morphogenesis of a fungus.

29. An antifungal agent obtained by the process of claim 24.

30. A process of validating specificity of an antifungal agent comprising:
(a) providing a candidate drug, and
(b) determining at least one activity of a fungal Bot1 polypeptide of claim 1 or variant thereof in the presence of the candidate drug,
(c) confirming that the candidate drug is specific for a fungus because of its ability to at least affect one activity of the fungal Bot1 polypeptide or variant thereof.
